(19) United States
(12) Patent Application Publication
(10) Pub. No.: US 2007/0225333 A1
(43) Pub. Date: Sep. 27, 2007

(54) 3-Heterocyclyl-4-Phenyl-Triazole Derivatives as Inhibitors of the Vasopressin Via Receptor

(76) Inventors: Justin S Bryans, Canterbury (GB); Patrick S. Johnson, Sandwich (GB); Thomas Ryckmans, Kent (GB); Alan Stobie, Sandwich (GB)

Correspondence Address: PFIZER INC., PATENT DEPARTMENT, MS8260-1611, EASTERN POINT ROAD, GROTON, CT, 06340, US

(21) Appl. No.: 10/598,840

(22) PCT Filed: Apr. 18, 2005
(86) PCT No.: PCT/IB05/01062

§ 371 Date: May 18, 2007

Related U.S. Application Data
(60) Provisional application No. 60/576,252, filed on Jun. 2, 2004.
(60) Provisional application No. 60/601,082, filed on Aug. 12, 2004.

(30) Foreign Application Priority Data
Apr. 28, 2004 (GB) 0409502.2
Jul. 15, 2004 (GB) 0415888.7

(51) Int. Cl. C07D 401/04; C07D 401/14; C07D 403/04

(52) U.S. Cl. 514/336; 514/383; 546/272.7; 548/262.2

(57) ABSTRACT

Compounds of formula (I): or a pharmaceutically acceptable derivative thereof, wherein R represents C₁₋₆alkyl (optionally substituted by C₁₋₆alkyloxy or Het) or C₁₋₆alkyloxy; R¹ and R² independently represent hydrogen, halo or C₁₋₆alkyl, ring A represents Het¹; X represents O or NR³; R³ represents hydrogen or C₁₋₆alkyl; ring B represents a phenyl group or Het², either of which may be optionally substituted with one or more groups selected from halo, CN, C₁₋₆alkyloxy, CF₃, C₁₋₆alkyl, NH₂ and NO₂; Het and Het¹ independently represent a 5- or 6-membered saturated, partially unsaturated or aromatic heterocyclic group comprising either (a) 1 to 4 nitrogen atoms, (b) one oxygen or one sulphur atom or (c) 1 oxygen atom or 1 sulphur atoms and 1 or 2 nitrogen atoms are useful for treating anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis) mittlesmerchz, preclampsia, premature ejaculation, premature (preterm) labour and Raynaud's disease.

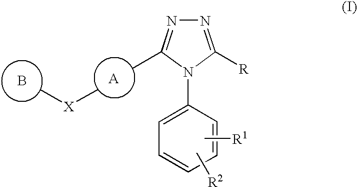

This invention relates to triazole derivatives. It also relates to their uses, processes for their preparation, the intermediates used in their preparation and compositions containing them.

The compounds of the present invention are indicated in the treatment of a wide range of disorders, particularly aggression, Alzheimer's disease, anorexia nervosa, anxiety, anxiety disorder, asthma, atherosclerosis, autism, cardiovascular disease (including angina, atherosclerosis, hypertension, heart failure, edema, hypernatremia), cataract, central nervous system disease, cerebrovascular ischemia, cirrhosis, cognitive disorder, Cushing's disease, depression, diabetes mellitus, dysmenorrhoea (primary and secondary), emesis (including motion sickness), endometriosis, gastrointestinal disease, glaucoma, gynaecological disease, heart disease, intrauterine growth retardation, inflammation (including rheumatoid arthritis), ischemia, ischemic heart disease, lung tumour, micturition disorder, mittlesmerchz, neoplasm, nephrotoxicity, non-insulin dependent diabetes, obesity, obsessive/compulsive disorder, ocular hypertension, preclampsia, premature ejaculation, premature (preterm) labour, pulmonary disease, Raynaud's disease, renal disease, renal failure, male or female sexual dysfunction, septic shock, sleep disorder, spinal cord injury, thrombosis, urogenital tract infection or urolithiasis.

Particularly of interest are the following diseases or disorders: anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittlesmerchz, preclampsia, premature ejaculation, premature (preterm) labour and Raynaud's disease.

Notably, the compounds of the present invention are useful in the treatment of dysmenorrhoea (primary and secondary).

There is a high unmet need in the area of menstrual disorders and it is estimated that up to 90% of all menstruating women are affected to some degree. Up to 42% of women miss work or other activities due to menstrual pain and it has been estimated that around 600 million work hours a year are lost in the US as a result {Coco, A. S. (1999). Primary dysmenorrhoea. [Review] [30 refs]. American Family Physician, 60, 489-96.}.

Menstrual pain in the lower abdomen is caused by myometrial hyperactivity and reduced uterine blood flow. These pathophysiological changes result in abdominal pain that radiates out to the back and legs. This may result in women feeling nauseous, having headaches and suffering from insomnia. This condition is called dysmenorrhoea and can be classified as either primary or secondary dysmenorrhoea.

Primary dysmenorrhoea is diagnosed when no abnormality causing the condition is identified. This affects up to 50% of the female population {Coco, A. S. (1999). Primary dysmenorrhoea. [Review] [30 refs]. American Family Physician, 60, 489-96.; Schroeder, B. & Sanfilippo, J. S. (1999). Dysmenorrhoea and pelvic pain in adolescents. [Review] [78 refs]. Pediatric Clinics of North America, 46, 555-71}. Where an underlying gynaecological disorder is present, such as endometriosis, pelvic inflammatory disease (PID), fibroids or cancers, secondary dysmenorrhoea will be diagnosed. Secondary dysmenorrhoea is diagnosed in only approximately 25% of women suffering from dysmenorrhoea. Dysmenorrhoea can occur in conjunction with menorrhagia, which accounts for around 12% of referrals to gynaecology outpatients departments.

Currently, women suffering from primary dysmenorrhoea are treated with non-steroidal anti-inflammatory drugs (NSAID's) or the oral contraceptive pill. In cases of secondary dysmenorrhoea surgery may be undertaken to correct the underlying gynaecological disorder.

Women suffering from dysmenorrhoea have circulating vasopressin levels which are greater than those observed in healthy women at the same time of the menstrual cycle. Inhibition of the pharmacological actions of vasopressin, at the uterine vasopressin receptor, may treat or alleviate the symptoms of dysmenorrhoea.

The compounds of the invention, and their pharmaceutically acceptable salts and solvates, have the advantage that they are selective inhibitors of the V1a receptor (and so are likely to have reduced side effects), they may have a more rapid onset of action, they may be more potent, they may be longer acting, they may have greater bioavailability or they my have other more desirable properties than the compounds of the prior art.

The invention therefore provides a compound of the formula (I): 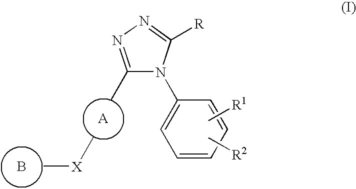
or a pharmaceutically acceptable derivative thereof, wherein
R represents C₁₋₆alkyl (optionally substituted by C₁₋₆alkyloxy or Het), or C₁₋₆alkyloxy;
R¹ and R² independently represent hydrogen, halo or C₁₋₆alkyl;
ring A represents Het¹;
X represents O or NR³;
   R³ represents hydrogen or C₁₋₆alkyl;
ring B represents a phenyl group or Het², either of which may be optionally substituted with one or more groups selected from halo, CN, C₁₋₆alkyloxy, CF₃, C₁₋₆alkyl, NH₂ and NO₂;
   Het, and Het¹ independently represent a 5- or 6-membered saturated, partially unsaturated or aromatic heterocyclic group comprising either (a) 1 to 4 nitrogen atoms, (b) one oxygen or one sulphur atom or (c) 1 oxygen atom or 1 sulphur atoms and 1 or 2 nitrogen atoms;
   Het² represents a 5- or 6-membered aromatic heterocyclic group comprising either (a) 1 to 4 nitrogen atoms, (b) one oxygen or one sulphur atom or (c) 1 oxygen atom or 1 sulphur atoms and 1 or 2 nitrogen atoms.

In the above definitions, halo means fluoro, chloro, bromo or iodo. Alkyl alkylene and alkyloxy groups, containing the requisite number of carbon atoms, can be unbranched or branched. Examples of alkyl include methyl, ethyl, n-propyl, i-propyl, n-butyl, i-butyl, sec-butyl and t-butyl. Examples of alkyloxy include methoxy, ethoxy, n-propoxy, i-propoxy, n-butoxy, i-butoxy, sec-butoxy and t-butoxy. Examples of alkylene include methylene, 1,1-ethylene, 1,2-ethylene, 1,1-propylene, 1,2-propylene, 1,3-propylene and 2,2-propylene. Het represents a heterocyclic group, examples of which include tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, 3,4-dihydro-2H-pyranyl, 5,6-dihydro-2H-pyranyl, 2H-pyranyl, 1,2,3,4-tetrahydropyridinyl, 1,2,5,6-tetrahydropyridinyl, pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl and pyrazinyl.

Preferred aspects of the invention are selected from any one or more of those described below:
1. R represents C₁₋₆alkyl, preferably methyl;
2. R represents C₁₋₆alkyloxy, preferably methoxy;
3. R represents C₁₋₆alkylene-oxy-C₁₋₆alkyl, preferably methoxymethylene or ethoxymethylene;
4. R represents C₁₋₆alkylene-Het, preferably methylene-Het, and Het is preferably triazolyl, morpholinyl or piperidinyl;
5. R¹ represents halo, preferably chloro;
6. R² represents hydrogen or methyl;
7. ring A is attached to the triazole ring via a nitrogen atom;
8. ring A represents piperidinylene;
9. X represents O;
10. NR³ represents NH or NMe;
11. ring B represents a phenyl, pyridinyl or pyrazinyl group;
12. ring B is substituted, preferably mono- or di-substituted, preferably the substituents is selected from F, Cl, CN, methyl, methoxy, CF₃, NO₂, CONH₂;

Specific preferred compounds according to the invention are those listed in the Examples section below, and the pharmaceutically acceptable salts thereof. In particular:
1-[4-(4-Chloro-phenyl)-5-methyl-4H-[1,2,4]triazole-3-yl]-4-phenoxy-piperidine;
2-({1-[4-(4-Chlorophenyl)-5-(methoxymethyl)-4H-1,2,4-triazol-3-yl]piperidin-4-yl}oxy)pyridine;
2-{1-[4-(4-Chloro-phenyl)-5-[1,2,3]triazole-2-ylmethyl-4H-[1,2,4]triazole-3-yl]-piperidin-4-yloxy}-pyrimidine;
2-{1-[4-(4-Chloro-phenyl)-5-ethoxy-4H-[1,2,4]triazole-3-yl]-piperidin-4-yloxy}-pyrimidine;
N-{1-[4-(4-Chlorophenyl)-5-methyl-4H-1,2,4-triazol-3-yl]-piperidin-4-yl}-N-methylpyridin-2-amine; and
N-{1-[4-(4-Chlorophenyl)-5-methyl-4H-1,2,4-triazol-3-yl]-piperidin-4-yl}-N-methylpyrimidin-2-amine

Pharmaceutically acceptable derivatives of the compounds of formula (I) according to the invention include salts, solvates, complexes, polymorphs, prodrugs, stereoisomers, geometric isomers, tautomeric forms, and isotopic variations of compounds of formula (I). Preferably, pharmaceutically acceptable derivatives of compounds of formula (I) comprise salts, solvates, esters and amides of the compounds of formula (I). More preferably, pharmaceutically acceptable derivatives of compounds of formula (I) are salts and solvates.

Pharmaceutically acceptable salts of the compounds of formula (I) include the acid addition and base salts thereof.

Suitable acid addition salts are formed from acids that form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components that may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975).

Hereinafter all references to compounds of formula (I) include references to salts, solvates and complexes thereof and to solvates and complexes of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include:
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counter-ion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, Stereochemistry of Organic Compounds by E. L. Eliel and S. H. Wilen (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹³O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The compounds of the invention are useful in therapy. Therefore, a further aspect of the invention is the use of a compound of formula (I), or a pharmaceutically salt or solvate thereof, as a medicament.

The compounds of the invention show activity as V1a antagonists. Therefore, a further aspect of the invention is the method of treatment of a mammal, including a human being, to treat a disorder for which a V1a antagonist is indicated, comprising administering a therapeutically effective amount of a compound of formula (I), or a pharmaceutically acceptable salt or solvate thereof, to the mammal. In particular, the compounds of formula (I) are useful in treating anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittlesmerchz, preclampsia, premature ejaculation, premature (preterm) labour or Raynaud's disease. Even more particularly, they are useful in treating dysmenorrhoea (primary or secondary).

A further aspect of the present invention is the use of a compound of formula (I), or a pharmaceutically acceptable salt or solvate thereof, in the manufacture of a medicament for the treatment of a disorder for which a V1a receptor antagonist is indicated.

All of the compounds of the formula (I) can be prepared by the procedures described in the general methods presented below or by the specific methods described in the Examples section and the Preparations section, or by routine modifications thereof. The present invention also encompasses any or one or more of these processes for preparing the compounds of formula (I), in addition to any novel intermediates used therein.

In the following general methods, R, R¹, R², R³, ring A, ring B, X, Het, Het¹ and Het² are as previously defined for a compound of the formula (I) unless otherwise stated.

Compounds of general formula (I), where X represents NR⁴ or O and R¹, R² and R³ are as previously defined for a compound of the formula (I), may be prepared according to reaction Scheme 1.

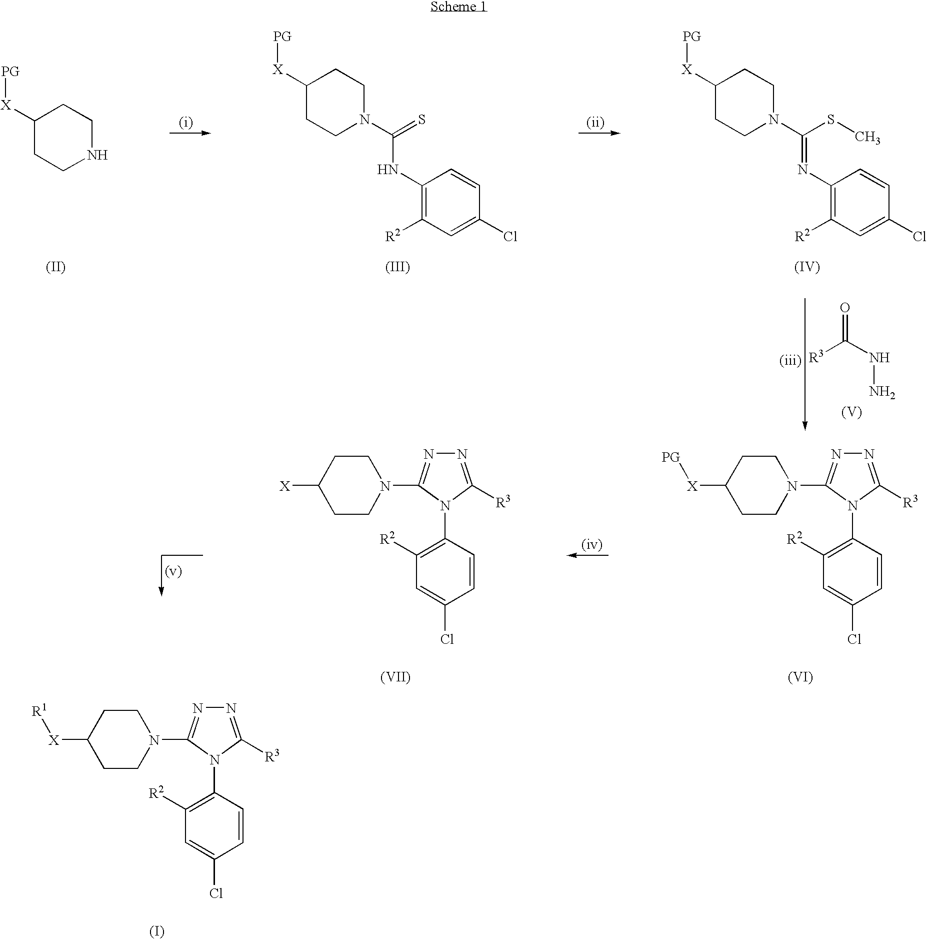

PG is a suitable N or O protecting group. Typically PG is a carbamate, preferably a Boc group when X represents N. Typically PG is an ester group, preferably an acetate when X represents O.

When X represents NH and PG represents Boc, compounds of general formula (II) are commercially available.

When X represents O and PG represents C(O)Me, compounds of general formula (II) can be prepared as described in J. Org. Chem. 68(2), 613; 2003.

Compounds of general formula (III) can be prepared from compounds of general formula (II) by step (i) in Scheme I above: i.e., reaction with a suitably substituted aromatic isothiocyanate (R²ClPhNCS), in a suitable solvent such as methanol or ethanol at an ambient temperature for between 1 to 6 hours. Typical conditions comprise 1.0 equivalent of compound (II) and 1.0 equivalent of R²ClPhNCS in ethanol at room temperature for 1 to 6 hours.

Compounds of general formula (IV) can be prepared from compounds of general formula (III) by step (ii) in Scheme 1 above: i.e. alkylation with a suitable alkylating agent, such as methyl tosylate or methyl iodide, in the presence of a suitable base, such as potassium tert-butoxide or potassium carbonate, in a suitable solvent, such as tetrahydrofuran or ethanol, under ambient conditions for 1 to 4 hours. Typical conditions comprise 1.0 equivalent of compound (III), 1.0 equivalent of potassium tert-butoxide and 1.0 equivalent of methyl tosylate in tetrahydrofuran, at room temperature for 3 hours.

Compounds of general formula (V) are either commercially available or can be prepared from esters of formula R³C(O)OR⁵, wherein R⁵ is a C₁-C₄ alkyl group, preferably a methyl or ethyl group, such that the OR⁵ group may be displaced by hydrazine to form the compound of formula (V), using standard methodology as exemplified in preparations 32 and 33.

Compounds of general formula (VI) can be prepared from compounds of formula (IV) and (V) by step (iii) in Scheme 1 above: i.e. reaction with a suitable hydrazide (R³C(O)NHNH₂ (V)), optionally in the presence of an acid catalyst such as para-toluenesulfonic acid or trifluoroacetic acid, in a suitable solvent, such as “butanol or tetrahydrofuran, at an elevated temperature for 2 to 8 hours. Typical conditions comprise 1.0 equivalent of compound (IV), 1.0 to 1.5 equivalents of hydrazide and 0.5 equivalents of trifluoroacetic acid, in tetrahydrofuran, heated under reflux for 5 hours.

Optionally, when X represents NR⁴ and R⁴ represents alkyl, compounds of formula (VI) can be converted into alternative compounds of general formula (VI) by N-alkylation using a suitable alkylating agent, such as R⁴Y where Y represents Hal and is preferably iodide, in the presence of a suitable base, such as sodium hydride or potassium tert-butoxide, in a suitable solvent, such as tetrahydrofuran, at an ambient temperature for 1 to 6 hours. Typical conditions comprise 1.0 equivalent of compound (V), 5.0 equivalents of sodium hydride and 2.0 equivalents of methyl iodide in tetrahydrofuran, at room temperature for 3 hours.

Compounds of general formula (VII) can be prepared from compounds of general formula (VI) by step (iv) in Scheme 1 above: i.e. de-protection of compound (VI). This may be achieved using standard methodology as described in “Protecting Groups in Organic Synthesis” by T. W. Greene and P. Wutz.

When PG represents Boc, typical conditions comprise 1.0 equivalent of compound (VI) in the presence of hydrochloric acid (4M in dioxan), in dichloromethane, at room temperature for 18 hours.

Alternatively, when PG represents C(O)CH₃, typical conditions comprise 1.0 equivalent of compound (VI) and 3.0 equivalents of potassium carbonate (1M in methanol) at room temperature for 4 hours.

Compounds of general formula (I) may be prepared from compounds of formula (VII) by step (v) in Scheme I above:

When X represents O, this may be achieved by a Mitsunobu reaction with a suitable phenol (R¹—OH) in the presence of a suitable phosphine, such as tri-ⁿbutyl phosphine or triphenyl phosphine, and a suitable azo compound, such as di-tert-butyl azodicarboxylate or 1′1′-azobis(N,N-dimethylformamide), in a suitable solvent, such as dichloromethane, tetrahydrofuran or N,N-dimethylformamide, at an ambient temperature for 1 to 8 hours. Typical conditions comprise 1.0 equivalent of compound (VII), 2.0 equivalents of R¹—OH, 1.0 to 1.2 equivalents of triphenyl phosphine and 2.0 equivalents of di-tert-butyl azodicarboxylate in dichloromethane, at room temperature for 3 hours.

When X represents NR⁴, compounds of general formula (I) may be prepared by N-alkylation using a suitable alkylating agent (R¹Z where Z represents chloro or bromo) in the presence of a suitable base, such as triethylamine or N,N-diisopropylethylamine, in a suitable solvent, such as ethanol or tetrahydrofuran, at a temperature of between 25° C. to 85° C., for 18 to 48 hours. Typical conditions comprise 1.0 equivalent of compound (VI), 1.0 equivalent of R¹-Z and 3.0 equivalents of triethylamine in tetrahydrofuran, heated under reflux for 18 hours.

Compounds of formula (I) may be converted to alternative compounds of formula (I) using standard chemical transformations as exemplified in examples 46 and 47.

Compounds of formula (I) may alternatively be prepared according to reaction Scheme 2.

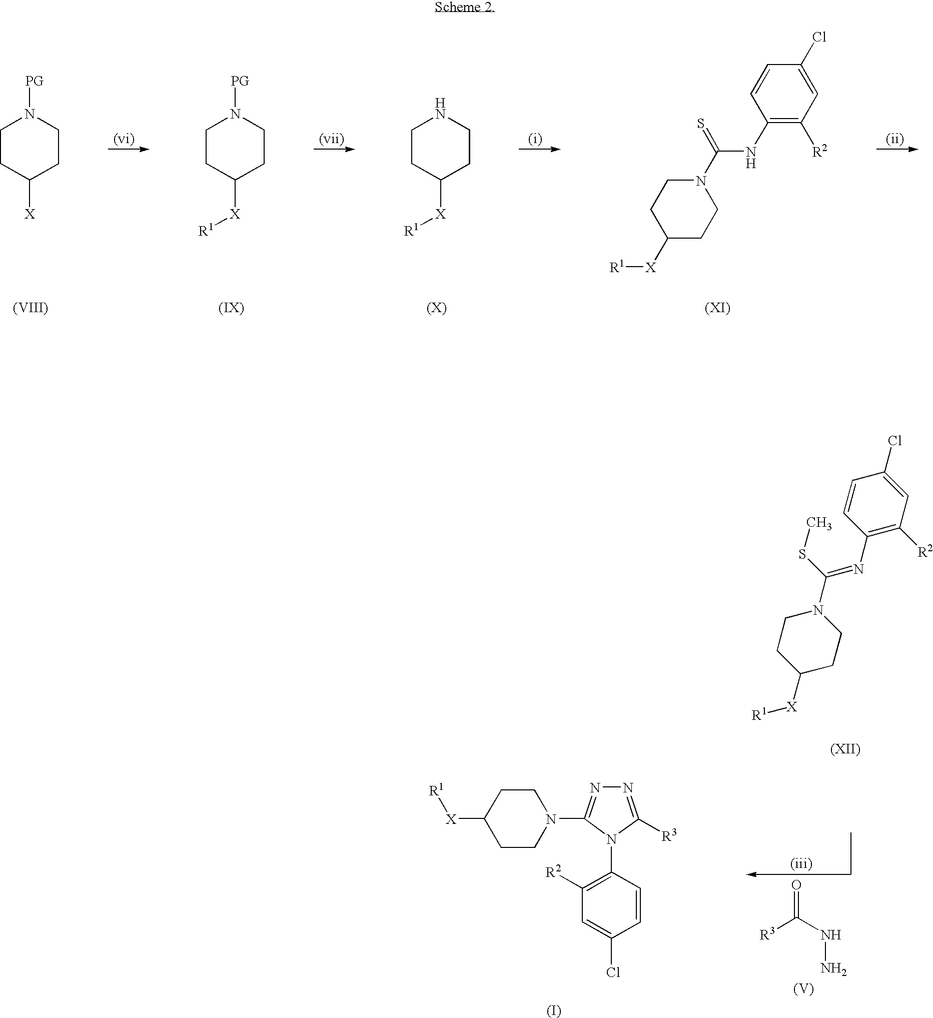

PG is a suitable N protecting group, typically a carbamate, preferably a Boc group.

When PG represents benzyl or BOC, compounds of general formula (VIII) are commercially available.

Compounds of general formula (IX) can be prepared from compounds of general formula (VIII) by step (vi) in Scheme 2 above: i.e. alkylation of compound (VIII) with a suitable alkylating agent (R¹-Z), where Z represents chloro or bromo.

When X represents O, the reaction is carried out in the presence of a suitable base, such as sodium hydride or potassium tert-butoxide, in a suitable solvent, such as 1-methyl-2-pyrrolidinone, at an elevated temperature for 1 to 8 hours. Typical conditions comprise 1.0 equivalent of compound (VIII), 1.0 equivalent of het-Z and 1.0 equivalent of sodium hydride in 1-methyl-2-pyrrolidinone, heated under reflux for 6 hours.

When X represents NR⁴, the reaction is carried out in the presence of a suitable base, such as sodium tert-butoxide or potassium tert-butoxide, a suitable catalyst, such as tris(dibenzylideneacetone)dipalladium(0), and a chelating ligand, such as bis(diphenylphosphino)propane, in a suitable solvent, such as ethanol or toluene, at an elevated temperature for 6 to 24 hours. Typical conditions comprise 1.0 equivalent of compound (VIII), 1.0 to 1.5 equivalents of sodium tert-butoxide, 1.0 equivalent of R¹-Z, tris(dibenzylideneacetone)dipalladium(0) (cat.) and bis(diphenylphosphino)-propane in ethanol, heated under reflux for 18 hours.

Compounds of general formula (X) can be prepared from compounds of general formula (IX) by step (vii) in Scheme 2 above: i.e. de-protection of compound (IX). This may be achieved using standard methodology as described in “Protecting Groups in Organic Synthesis” by T. W. Greene and P. Wutz.

When PG represents Boc, typical conditions comprise 1.0 equivalent of compound (IX) in the presence of hydrochloric acid (4M in dioxan), in dichloromethane, at room temperature for 1 to 3 hours.

Alternatively, when PG represents benzyl, typical conditions comprise 1.0 equivalent of compound (IX) and 10% palladium hydroxide (cat.) in ethanol, under 60 psi of hydrogen, heated under reflux for 2 to 8 hours.

Compounds of formula (XI) can be prepared from compounds of formula (X) by step (i) as described in Scheme 1 above.

Compounds of formula (XII) can be prepared from compounds of formula (XI) by step (ii) as described in Scheme 1 above.

Compounds of formula (I) can be prepared from compounds of formula (XII) by step (iii) as described in Scheme 1 above.

Compounds of general formula (I) where X represents O, R³ represents CH₃ and R¹, R² and R³ are as described herein, may alternatively be prepared according to reaction Scheme 3.

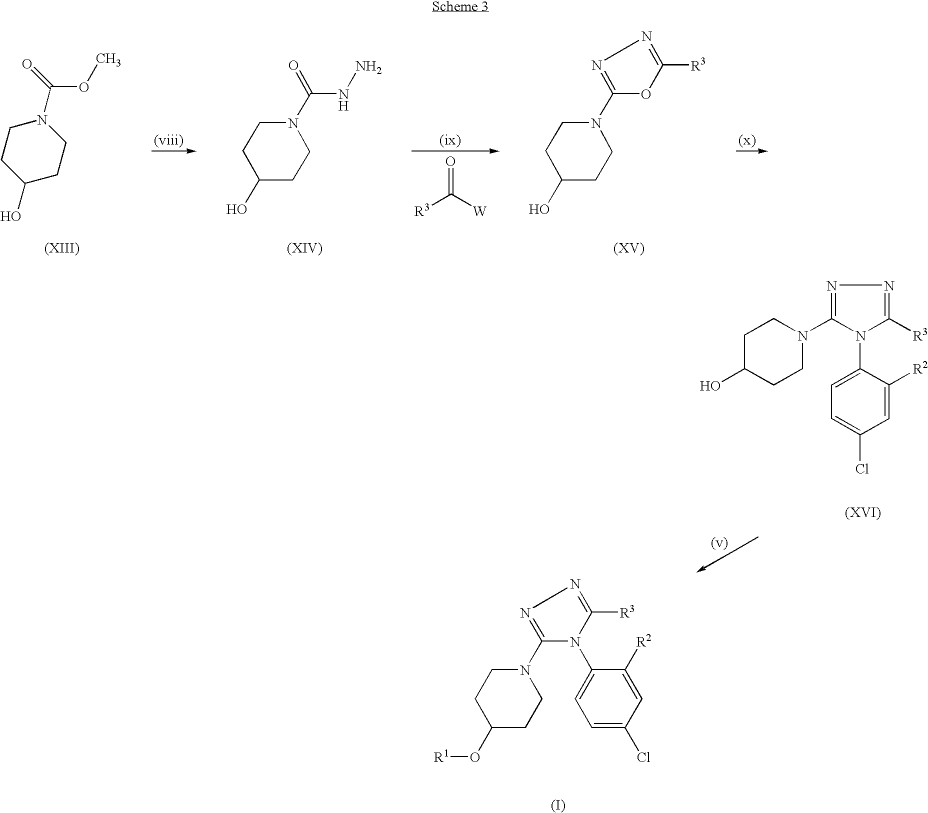

Compounds of general formula (XIII) can be prepared by analogy with the method of E. Falch et al (Eur. J. Med. Chem. 26(1), 69-77; 1991).

Compounds of general formula (XIV) can be prepared from compounds of formula (XIII) by step (viii) in Scheme 3 above: i.e. reaction with hydrazine monohydrate in a suitable solvent such as methanol or ethanol at elevated temperature for 1 to 48 hours.

Compounds of general formula (XV) can be prepared from compounds of general formula (XIV) by step (ix) in Scheme 3 above: i.e. a coupling reaction with a suitable acetylating agent (R³C(O)W wherein W represents OH or Cl), followed by cyclisation under suitable dehydration conditions.

When W represents Cl, coupling is carried out in dichloromethane or tetrahydrofuran, optionally in the presence of base, such as triethylamine, Hünig's base or N-methylmorpholine, at an ambient temperature for 1 to 24 hours, followed by cyclisation using a suitable dehydration reagent, such as polyphosphoric acid, phosphorous oxychloride, or triflic anhydride with pyridine, optionally in a suitable solvent, such as dichloromethane, at temperatures of between 50 to 120° C. for 5 minutes to 12 hours.

When W represents OH, coupling is carried out in the presence of a conventional coupling agent, such as WSCDI /DCC or HOBT /HOAT, optionally in the presence of a catalyst, with an excess of acid acceptor, such as N-methylmorpholine, triethylamine or Hunig's base, in a suitable solvent such as tetrahydrofuran, dichloromethane or ethyl acetate, at an ambient temperature for 4 to 24 hours. This step is followed by cyclisation using a suitable dehydration reagent such as polyphosphoric acid, phosphorous oxychloride, or triflic anhydride with pyridine, optionally in a suitable solvent, such as dichloromethane, at temperatures between 50 and 120° C., for between 5 minutes to 12 hours.

Alternatively, when R³ represents Me, N,N-dimethylacetamide dimethyl acetal (ex Aldrich) is used as the most preferable acetylating agent, optionally in the presence of a base, such as such as triethylamine, N-methylmorpholine, or sodium carbonate, in a suitable solvent, such as N,N-dimethylformamide, N-methyl pyrrolidine or toluene, followed by the addition of a suitable acid catalyst, such as trifluoroacetic acid, para-toluenesulfonic acid, camphor sulfonic acid or hydrochloric acid, at an elevated temperature for 1 to 8 hours.

Compounds of general formula (XVI) can be prepared from compounds of formula (XV) by step (x) in Scheme 3 above: i.e. reaction with a suitably substituted chloro aniline (R²ClPh—NH₂) in the presence of a suitable acid catalyst, such as trifluoroacetic acid, para-toluenesulfonic acid, camphor sulfonic acid or hydrochloric acid, in a suitable solvent, such as xylene or toluene, heated at elevated temperature for 1 to 48 hours.

Compounds of general formula (I) may be prepared from compounds of general formula (XVI) by step (v) in Scheme 3 above: i.e. a Mitsunobu reaction as described in Scheme 1.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

Compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

A further aspect of the invention is a pharmaceutical formulation including a compound of formula (I), or a pharmaceutically acceptable, salt or solvate thereof, together with a pharmaceutically acceptable excipient, diluent or carrier. In a further embodiment there is provided the pharmaceutical formulation for administration either prophylactically or when pain commences.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve, which delivers a metered amount. The overall daily dose will typically be in the range 0.01 μg to 15 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.01 mg to 15 mg depending, of course, on the mode of administration. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The compounds of the present invention may be tested in the screens set out below:

1.0 V_{1A} Filter Binding Assay

1.1 Membrane Preparation

Receptor binding assays were performed on cellular membranes prepared from CHO cells stably expressing the human V_{1A} receptor, (CHO-hV_{1A}). The CHO-hV_{1A} cell line was kindly provided under a licensing agreement by Marc Thibonnier, Dept. of Medicine, Case Western Reserve University School of Medicine, Cleveland, Ohio. CHO-hV_{1A} cells were routinely maintained at 37° C. in humidified atmosphere with 5% CO₂ in DMEM/Hams F12 nutrient mix supplemented with 10 % fetal bovine serum, 2 mM L-glutamine, 15 mM HEPES and 400 μg/ml G418. For bulk production of cell pellets, adherent CHO-hV_{1A} cells were grown to confluency of 90-100% in 850 cm² roller bottles containing a medium of DMEM/Hams F12 Nutrient Mix supplemented with 10% fetal bovine serum, 2 mM L-glutamine and 15 mM HEPES. Confluent CHO-hV_{1A} cells were washed with phosphate-buffered saline (PBS), harvested into ice cold PBS and centrifuged at 1,000 rpm. Cell pellets were stored at −80° C. until use. Cell pellets were thawed on ice and homogenised in membrane preparation buffer consisting of 50 mM Tris-HCl, pH 7.4, 5 mM MgCl₂ and supplemented with a protease inhibitor cocktail, (Roche). The cell homogenate was centrifuged at 1000 rpm, 10 min, 4° C. and the supernatant was removed and stored on ice. The remaining pellet was homogenised and centrifuged as before. The supernatants were pooled and centrifuged at 25,000× g for 30 min at 4° C. The pellet was resuspended in freezing buffer consisting of 50 mM Tris-HCl, pH 7.4, 5 mM MgCl₂ and 20% glycerol and stored in small aliquots at −80° C. until use. Protein concentration was determined using Bradford reagent and BSA as a standard.

1.2 V_{1A} Filter Binding

Protein linearity followed by saturation binding studies were performed on each new batch of membrane. Membrane concentration was chosen that gave specific binding on the linear portion of the curve. Saturation binding studies were then performed using various concentrations of [³H]-arginine vasopressin, [³H]-AVP (0.05 nM-100 nM) and the K_{d} and Bₘₐₓ determined.

Compounds were tested for their effects on [³H]-AVP binding to CHO-hV_{1A} membranes, (³H-AVP; specific activity 65.5 Ci/mmol; NEN Life Sciences). Compounds were solubilised in dimethylsulfoxide (DMSO) and diluted to working concentration of 10% DMSO with assay buffer containing 50 mM Tris-HCL pH 7.4, 5 mM MgCl₂ and 0.05% BSA. 25 μl compound and 25 μl [³H]-AVP, (final concentration at or below K_{d} determined for membrane batch, typically 0.5 nM-0.6 nM) were added to a 96-well round bottom polypropylene plate. The binding reaction was initiated by the addition of 200 μl membrane and the plates were gently shaken for 60 min at room temperature. The reaction was terminated by rapid filtration using a Filtermate Cell Harvester (Packard Instruments) through a 96-well GF/B UniFilter Plate which had been pre-soaked in 0.5% polyethyleneimine to prevent peptide sticking. The filters were washed three times with 1 ml ice cold wash buffer containing 50 mM Tris-HCL pH 7.4 and 5 mM MgCl₂. The plates were dried and 50 μl Microscint-0 (Packard instruments) was added to each well. The plates were sealed and counted on a TopCount Microplate Scintillation Counter (Packard Instruments). Non-specific binding (NSB) was determined using 1 μM unlabelled d(CH2)5Tyr(Me)AVP ([β-mercapto-β,β-cyclopentamethylenepropionyl,0-Me-Tyr²,Arg⁸]-vasopressin) (βMCPVP), (Sigma). The radioligand binding data was analysed using a four parameter logistic equation with the min forced to 0%. The slope was free fitted and fell between −0.75 and −1.25 for valid curves. Specific binding was calculated by subtracting the mean NSB cpm from the mean Total cpm. For test compounds the amount of ligand bound to the receptor was expressed as % bound=(sample cpm−mean NSB cpm)/specific binding cpm ×100. The % bound was plotted against the concentration of test compound and a sigmoidal curve was fitted. The inhibitory dissociation constant (Kᵢ) was calculated using the Cheng-Prusoff equation: Kᵢ=IC₅₀/(1+[L]/K_{d}) where [L] is the concentration of ligand present in the well and K_{d} is the dissociation constant of the radioligand obtained from Scatchard plot analysis.

2.0 V_{1A} Functional Assay; Inhibition of AVP/V_{1A}-R Mediated Ca²⁺ Mobilization by FLIPR (Fluorescent Imaging Plate Reader) (Molecular Devices)

Intracellular calcium release was measured in CHO-hV_{1A} cells using FLIPR, which allows the rapid detection of calcium following receptor activation. The CHO-hV_{1A} cell line was kindly provided under a licensing agreement by Marc Thibonnier, Dept. of Medicine, Case Western Reserve University School of Medicine, Cleveland, Ohio. CHO-V_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. On the afternoon before the assay cells were plated at a density of 20,000 cells per well into black sterile 96-well plates with clear bottoms to allow cell inspection and fluorescence measurements from the bottom of each well. Wash buffer containing Dulbecco's phosphate buffered saline (DPBS) and 2.5 mM probenecid and loading dye consisting of cell culture medium containing 4 μM Fluo-3-AM (dissolved in DMSO and pluronic acid),(Molecular Probes) and 2.5 mM probenecid was prepared fresh on the day of assay. Compounds were solubilised in DMSO and diluted in assay buffer consisting of DPBS containing 1% DMSO, 0.1% BSA and 2.5 mM probenecid. The cells were incubated with 100 μl loading dye per well for 1 hour at 37° C. in humidified atmosphere with 5% CO₂. After dye loading the cells were washed three times in 100 μl wash buffer using a Denley plate washer. 100 μl wash buffer was left in each well. Intracellular fluorescence was measured using FLIPR. Fluorescence readings were obtained at 2s intervals with 50 μl of the test compound added after 30s. An additional 155 measurements at 2s intervals were then taken to detect any compound agonistic activity. 50 μl of arginine vasopressin (AVP) was then added so that the final assay volume was 200 μl. Further fluorescence readings were collected at 1s intervals for 120s. Responses were measured as peak fluorescence intensity (FI). For pharmacological characterization a basal FI was subtracted from each fluorescence response. For AVP dose response curves, each response was expressed as a % of the response to the highest concentration of AVP in that row. For IC₅₀ determinations, each response was expressed as a % of the response to AVP. IC₅₀ values were converted to a modified K_{b} value using the Cheng-Prusoff equation which takes into account the agonist concentration, [A], the agonist EC₅₀ and the slope: K_{b}=IC₅₀/(2+[A]/A₅₀]ⁿ)^{1/n}−1 where [A] is the concentration of AVP, A₅₀ is the EC₅₀ of AVP from the dose response curve and n=slope of the AVP dose response curve.

The compounds of the invention may be administered alone or in combination with one or more other compounds of the invention or in combination with one or more other drugs (or as any combination thereof). Such combinations offer significant advantages, including synergistic activity, in therapy.

The compounds of the present invention may be administered in combination with an oral contraceptive. Thus in a further aspect of the invention, there is provided a pharmaceutical product containing an V1a antagonist and an oral contraceptive as a combined preparation for simultaneous, separate or sequential use in the treatment of dysmenorrhoea.

The compounds of the present invention may be administered in combination with a PDEV inhibitor. Thus in a further aspect of the invention, there is provided a pharmaceutical product containing a V1a antagonist and a PDEV inhibitor as a combined preparation for simultaneous, separate or sequential use in the treatment of dysmenorrhoea.

PDEV inhibitors useful for combining with V1a antagonists include, but are not limited to:
(i) The PDEV inhibitors mentioned in International Patent Application publication nos. WO03/000691; WO02/64590; WO02/28865; WO02/28859; WO02/38563; WO02/36593; WO02/28858; WO02/00657; WO02/00656; WO02/10166; WO02/00658; WO01/94347; WO01/94345; WO00/15639 and WO00/15228;
(ii) The PDEV inhibitors mentioned in U.S. Pat. Nos. 6,143,746; 6,143,747 and 6,043,252;
(iii) the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the hexahydropyrazino [2′,1′:6,1]pyrido [3,4-b]indole-1,4-diones disclosed in published international application WO95/19978; the pyrazolo [4,3-d]pyrimidin-4-ones disclosed in WO00/27848; the imidazo[5,1-f][1,2,4]triazin-ones disclosed in EP-A-1092719 and in published international application WO 99/24433 and the bicyclic compounds disclosed in published international application WO 93/07124; the pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719; the tricyclic compounds disclosed in EP-A-1241170; the alkyl sulphone compounds disclosed in published international application WO 02/074774; the compounds disclosed in published international application WO 02/072586; the compounds disclosed in published international application WO 02/079203 and the compounds disclosed in WO 02/074312.
(iv) Preferably 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);5-(2-ethoxy-5-morpholinoacetylphenyl)-1-methyl-3-n-propyl-1,6-dihydro-7H-pyrazolo[4,3-d]pyrimidin-7-one (see EP-A-0526004);3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one(see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one(see WO 01/27113, Example 15);5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)pyrazino[2′,1′:6,1]pyrido[3,4-b]indole-1,4-dione (tadalafil, IC-351, Cialis®), i.e. the compound of examples 78 and, 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®)) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433;the compound of example 11 of published international application WO93/07124 (EISAI); compounds 3 and 14 from Rotella D P, J. Med. Chem., 2000, 43, 1257; 4-(4-chlorobenzyl)amino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide.
(v) 4-bromo-5-(pyridylmethylamino)-6-(3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furaziocillin; cis-2-hexyl-5-methyl-3,4,5,6a,7,8,9,9a-octahydrocyclopent[4,5]-imidazo[2, 1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

The contents of the published patent applications and journal articles and in particular the general formulae of the therapeutically active compounds of the claims and exemplified compounds therein are incorporated herein in their entirety by reference thereto.

Preferably the PDEV inhibitor is selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one.

Most preferably the PDEV inhibitor is sildenafil and pharmaceutically acceptable salts thereof. Sildenafil citrate is a preferred salt.

The compounds of the present invention may be administered in combination with an NO donor. Thus in a further aspect of the invention, there is provided a pharmaceutical product containing a V1a antagonist and a NO donor as a combined preparation for simultaneous, separate or sequential use in the treatment of dysmenorrhoea.

The compounds of the present invention may be administered in combination with L-arginine, or as an arginate salt. Thus in a further aspect of the invention, there is provided a pharmaceutical product containing a V1a antagonist and L-arginine as a combined preparation for simultaneous, separate or sequential use in the treatment of dysmenorrhoea.

The compounds of the present invention may be administered in combination with a COX inhibitor. Thus in a further aspect of the invention, there is provided a pharmaceutical product containing a V1a antagonist and a COX inhibitor as a combined preparation for simultaneous, separate or sequential use in the treatment of dysmenorrhoea.

COX inhibitors useful for combining with the compounds of the present invention include, but are not limited to:
(i) ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester (See Wenk, et al, Europ. J Pharmacol. 453:319-324 (2002));
(ii) meloxicam, (CAS registry number 71125-38-7; described in U.S. Pat. No. 4,233,299), or a pharmaceutically acceptable salt or prodrug thereof;
(iii) Substituted benzopyran derivatives that are described in U.S. Pat. No. 6,271,253. Also benzopyran derivatives described in U.S. Pat. Nos. 6,034,256 and 6,077,850 along with International Publication No's WO 98/47890 and WO 00/23433;
(iv) Chromene COX2 selective inhibitors described in U.S. Pat. No. 6,077,850 and U.S. Pat. No. 6,034,256;
(v) The compounds described in International Patent Application Publication No's WO 95/30656, WO 95/30652, WO 96/38418 and WO 96/38442, and the compounds described in European Patent Application Publication No. 799823, along with the pharmaceutically acceptable derivatives thereof;
(vi) celecoxib (U.S. Pat. No. 5,466,823), valdecoxib (U.S. Pat. No. 5,633,272), deracoxib (U.S. Pat. No. 5,521,207), rofecoxib (U.S. Pat. No. 5,474,995), etoricoxib (International Patent Application Publication No. WO 98103484), JTE-522 (Japanese Patent Application Publication No. 9052882), or a pharmaceutically acceptable salt or prodrug thereof;
(vii) Parecoxib (described in U.S. Pat. No. 5,932,598), which is a therapeutically effective prodrug of the tricyclic Cox-2 selective inhibitor valdecoxib (described in U.S. Pat. No. 5,633,272), in particular sodium parecoxib;
(viii) ABT-963 (described in International Patent Application Publication No. WO 00/24719)
(ix) Nimesulide (described in U.S. Pat. No. 3,840,597), flosulide (discussed in J. Carter, Exp.Opin.Ther.Patents, 8(1), 21-29 (1997)), NS-398 (disclosed in U.S. Pat. No. 4,885,367), SD 8381 (described in U.S. Pat. No. 6,034,256), BMS-347070 (described in U.S. Pat. No. 6,180,651), S-2474 (described in European Patent Publication No. 595546) and MK-966 (described in U.S. Pat. No. 5,968,974);
(x) The compounds and pharmaceutically acceptable derivatives described in U.S. Pat. No. 6,395,724, U.S. Pat. No. 6,077,868, U.S. Pat. No. 5,994,381, U.S. Pat. No. 6,362,209, U.S. Pat. No. 6,080,876, U.S. Pat. No 6,133,292, U.S. Pat. No. 6, 369,275, U.S. Pat. No. 6,127,545, U.S. Pat. No. 6,130,334, U.S. Pat. No. 6,204,387, U.S. Pat. No. 6,071,936, U.S. Pat. No. 6,001,843, U.S. Pat. No. 6,040,450, International Patent Application Publication No WO 96/03392, International Patent Application Publication No WO 96/24585, U.S. Pat. No. 6,340,694, U.S. Pat. No. 6,376,519, U.S. Pat. No. 6,153,787, U.S. Pat. No. 6,046,217, U.S. Pat. No. 6,329,421, U.S. Pat. No. 6,239,137, U.S. Pat. No. 6,136,831, U.S. Pat. No. 6,297,282, U.S. Pat. No. 6,239,173, U.S. Pat. No. 6,303,628, U.S. Pat. No. 6,310,079, U.S. Pat. No. 6,300,363, U.S. Pat. No. 6,077,869, U.S. Pat. No. 6,140,515, U.S. Pat. No. 5,994,379, U.S. Pat. No. 6,028,202, U.S. Pat. No. 6,040,320, U.S. Pat. No. 6,083,969, U.S. Pat. No 6,306,890, U.S. Pat. No. 6,307,047, U.S. Pat. No. 6,004,948, U.S. Pat. No. 6,169,188, U.S. Pat. No. 6,020,343, U.S. Pat. No. 5,981,576, U.S. Pat. No. 6,222,048, U.S. Pat. No. 6,057,319, U.S. Pat. No. 6,046,236, U.S. Pat. No. 6,002,014, U.S. Pat. No. 5,945,539, U.S. Pat. No. 6,359,182, International Patent Application Publication No. WO 97/13755, International Patent Application Publication No. WO 96/25928, International Patent Application Publication No. WO 96/374679, International Patent Application Publication No. WO 95/15316, International Patent Application Publication No. WO 95/15315, International Patent Application Publication No. WO 96/03385, International Patent Application No. WO 95/00501, International Patent Application No. WO 94/15932, International Patent Application Publication No. WO 95/00501, International Patent Application Publication No. WO 94/27980, International Patent Application Publication No. WO 96/25405, International Patent Application Publication No. WO 96/03388, International Patent Application Publication No. WO 96/03387, U.S. Pat. No. 5,344,991, International Patent Application Publication No. WO 95/00501, International Patent Application Publication No. WO 96/16934, International Patent Application Publication No. WO 96/03392, International Patent Application Publication No. WO 96/09304, International Patent Application Publication No. WO 98/47890, and International Patent Application Publication No. WO 00/24719.

The contents of any of the patent applications, and in particular the general formulae of the therapeutically active compounds of the claims and exemplified compounds therein, are incorporated herein in their entirety by reference thereto.

The following Preparations and Examples illustrate the preparation of compounds of formula (I).

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCI). The following abbreviations have been used for common solvents: CDCl₃, deuterochloroform; D₆-DMSO, deuterodimethylsulphoxide; CD₃OD, deuteromethanol; THF, tetrahydrofuran. “Ammonia” refers to a concentrated solution of ammonia in water possessing a specific gravity of 0.88. Where thin layer chromatography (TLC) has been used it refers to silica gel TLC using silica gel 60 F254 plates, R_{f} is the distance travelled by a compound divided by the distance travelled by the solvent front on a TLC plate.

### Preparation 1: Piperidin-4-yl acetate

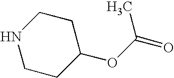

10% Pd/C (500 mg) was added to a solution of 1-benzylpiperidin-4-yl acetate [(10.7 g, 46 mmol), J. Org. Chem. 68(2), 613-616; 2003] in denatured ethanol (500 mL) and the mixture was stirred at 60° C., under 60 psi of hydrogen gas for 18 hours. The reaction mixture was then filtered through Arbocel®, washing through with ethanol, and the filtrate was concentrated in vacuo to afford the title product in quantitative yield, 7.2 g.

¹H NMR(CDCl₃, 400 MHz) δ: 1.60(m, 2H), 1.81-2.18(m, 5H), 2.78(m, 2H), 2.95-3.18(m, 2H), 4.82(m, 1H); MS APCI+ m/z 145 [MH]⁺

### Preparation 2: 1-{[(4-Chloro-2-methylphenyl)amino]carbonothioyl}piperidin-4-yl acetate

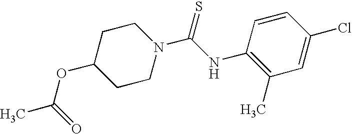

A solution of the product of preparation 1 (3.3 g, 23 mmol) in ethanol (15 mL) was added to a suspension of 4-chloro-2-methylphenyl isothiocyanate (4.2 g, 23 mmol) in ethanol (15 mL) and the mixture was stirred for 40 minutes at room temperature. The solvent was then evaporated under reduced pressure and the residue was dried in vacuo for 18 hours to afford the title compound in quantitative yield.

¹H NMR(CDCl₃, 400 MHz) δ: 1.75(m, 2H), 1.98(m, 2H), 2.10(s, 3H), 2.22(s, 3H), 3.72(m, 2H), 3.98(m, 2H), 5.13(m, 1H), 6.82(s, 1H), 7.05(d, 1H), 7.10-7.22(m, 2H).

### Preparation 3: 1-{[(4-Chlorophenyl)amino]carbonothioyl}piperidin-4-yl acetate

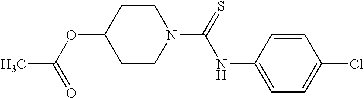

The title compound was prepared from the product of preparation 1 and 4-chlorophenyl isothiocyanate, using a similar method to that of preparation 2. Recrystallisation from toluene afforded the title compound as a solid in 81% yield.

¹H NMR(CDCl₃, 400MHz) δ: 1.75(m, 2H), 1.98(m, 2H), 2.10(s, 3H), 2.20(m, 1H), 3.77(m, 2H), 4.00(m, 2H), 5.03(m, 1H), 7.09(d, 1 H), 7.18(m, 1H), 7.31(m, 2H); MS APCI+ m/z 313 [MH]⁺

### Preparation 4: 1-[(4-Chloro-2-methylphenyl)imino](methylthio)methyl]-piperidin-4-yl acetate

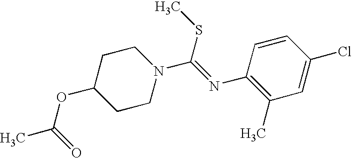

Potassium tert-butoxide (2.6 g, 23.2 mmol) was added to a suspension of the product of preparation 2 (7.52 g, 23.0 mmol) in tetrahydrofuran (75 mL) and the solution was stirred for 30 minutes at room temperature. Methyl tosylate (4.3 g, 23.2 mmol) was then added and the mixture was stirred 2 hours. Tlc analysis showed that not all of the starting material had been consumed and so further amounts of methyl tosylate (0.5 g, 2.7 mmol) and potassium tert-butoxide (0.1 g, 0.9 mmol) were added and the mixture was stirred for a further 30 minutes. The solvent was then evaporated under reduced pressure and the residue was partitioned between ethyl acetate and water. The aqueous layer was separated and extracted with ethyl acetate (×2). The combined organic solutions were dried over sodium sulfate and concentrated in vacuo. Purification of the residue by column chromatography on silica gel, eluting with pentane:dichloromethane, 80:20 to 0:100, afforded the title compound as a brown oil in 53% yield, 4.2 g.

¹H NMR(CDCl₃, 400 MHz) δ:′ 1.72(m, 2H), 1.98(m, 2H), 2.09(m, 9H), 3.39(m, 2H), 3.90(m, 2H), 5.01(m, 1H), 6.69(d, 1H), 7.05(d, 1H), 7.12(s, 1H); MS APCI+ m/z 341/343 [MH]⁺

### Preparation 5: 1-[(4-Chlorophenyl)imino](methylthio)methyl]piperidin-4-yl acetate

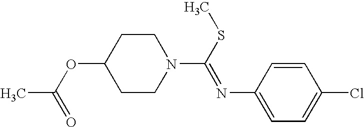

The title compound was prepared from the product of preparation 3, using a similar method to preparation 4, in quantitative yield.

¹H NMR(CDCl₃, 400MHz) δ: ¹HNMR(CDCl₃, 400MHz) δ: 1.71(m, 2H), 1.95(m, 2H), 2.05(s, 3H), 2.07(s, 3H), 3.40(m, 2H), 3.91(m, 2H), 5.00(m, 1H), 6.81(d, 2H), 7.22(d, 2H)

### Preparation 6: 1-[4-(4-Chloro-2-methylphenyl)-5-methyl-4H-1,2,4-triazol-3-yl]piperidin-4-yl acetate

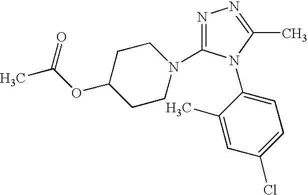

A mixture of the product of preparation 4 (4.2 g, 12.35 mmol) and acethydrazide (1.0 g, 13.6 mmol) in ⁿbutanol (40 mL) was heated under reflux for 18 hours. Further amounts of acethydrazide (2.0 g, 27.2 mmol) were then added at regular intervals until tlc analysis showed that all of the starting material had been consumed. The reaction mixture was then concentrated in vacuo and the residue was purified by column chromatography on silica gel, eluting with dichloromethane:methanol:0.88 ammonia, 100:0:0 to 95:5:0.5, to afford the title compound in 46% yield, 2.0 g.

¹H NMR(CDCl₃, 400 MHz) δ: 1.58(m, 2H), 1.78(m, 2H), 2.00-2.12(m, 9H), 2.90-3.03(m, 2H), 3.25(m, 2H), 4.84(m, 1H), 7.12(d, 1H), 7.32(d, 1H), 7.40(s, 1H); MS APCI+ m/z 349 [MH]⁺

### Preparation 7: 1-[4-(4-Chlorophenyl)-5-methyl-4H-1,2,4-triazol-3-yl]-piperidin-4-yl acetate

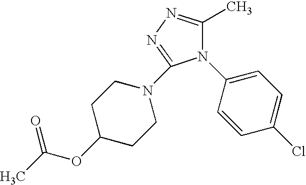

The title compound was prepared from the product of preparation 5, using a similar method to preparation 6, as a solid in 36% yield.

¹H NMR(CDCl₃, 400MHz) δ: 1.58(m, 2H), 1.77(m, 2H), 2.02(s, 3H), 2.25(s, 3H), 2.95(m, 2H), 3.23(m, 2H), 4.91(m, 1H), 7.28(d, 2H), 7.52(d, 2H); MS APCI+ m/z 335 [MH]⁺

### Preparation 8: 1-[4-(4-Chloro-2-methylphenyl)-5-methyl-4H-1,2,4-triazol-3-yl]piperidin-4-ol

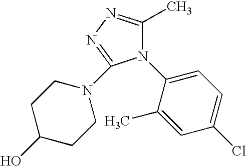

A mixture of the product of preparation 6 (2.79 g, 8 mmol) and potassium carbonate solution (1 M in methanol, 24 mL, 24 mmol) was stirred for 4 hours. The mixture was then evaporated under reduced pressure and the aqueous residue was diluted with ethyl acetate. The layers were separated and the organic layer was washed with water and brine, dried over sodium sulfate and concentrated in vacuo. Recrystallisation of the residue from ethyl acetate afforded the title compound as a solid in 77% yield, 1.9 g.

¹H NMR(CDCl₃, 400MHz) δ: 1.43(m, 2H), 1.80(m, 2H), 2.05-2.14(m, 6H), 2.80-2.97(m, 2H), 3.30(m, 2H), 3.78(m, 1H), 7.10(d, 1H), 7.33(d, 1H), 7.39(s, 1H); MS APCI+ m/z 307 [MH]⁺

### Preparation 9: 1-[4-(4-Chlorophenyl)-5-methyl-4H-1,2,4-triazol-3-yl]-piperidin-4-ol

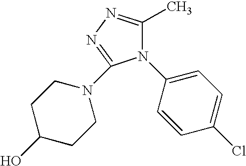

The title compound was prepared from the product of preparation 7, using a similar method to preparation 8, in 95% yield.

¹H NMR(CDCl₃, 400 MHz) δ: 1.48(m, 2H), 1.70(s, 3H), 1.82(m, 2H), 2.81(m, 2H), 3.25(m, 2H), 3.78(m, 1H), 7.29(d, 2H), 7.52(d, 2H); MS APCI+ m/z 293 [MH]⁺

### Preparation 10: tert-Butyl 4-(pyridin-2-yloxy)piperidine-1-carboxylate

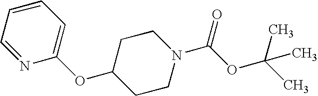

1-(tert-Butoxycarbonyl)-4-hydroxypiperidine (3.0 g, 14.9 mmol) was added to a suspension of sodium hydride (60% in mineral oil, 600 mg, 14.9 mmol) in 1-methyl-2-pyrrolidinone (50 mL) and the mixture was stirred at room temperature for 10 minutes. 2-Bromopyridine (1.43mL, 14.9mmol) was added and the mixture was heated under reflux for 6 hours. The mixture was then cooled to room temperature, diluted with ethyl acetate (250 mL) and washed with brine (5×150 mL). The organic solution was dried over magnesium sulfate and concentrated in vacuo. Purification of the residue by column chromatography on silica gel, eluting with pentane:ethyl acetate, 80:20, afforded the title compound as a colourless oil in 82% yield, 3.40 g.

¹H NMR(CDCl₃, 400 MHz) δ: 1.46(s, 9H), 1.71(m, 2H), 1.99(m, 2H), 3.28(m, 2H), 3.79(m, 2H), 5.22(m, 1H), 6.70(d, 1H), 6.86(m, 1H), 7.58(m, 1H), 8.12(d, 1H); MS APCI+ m/z 279 [MH]⁺

### Preparation 11: 2-(Piperidin-4-yloxy)pyridine dihydrochloride

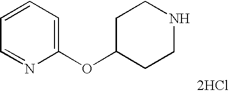

Hydrochloric acid (4M in dioxan, 20 mL) was added to a solution of the product of preparation 10 (3.36 g, 12.1 mmol), in methanol (3 mL), and the mixture was stirred for 30 minutes at room temperature. The reaction mixture was then concentrated in vacuo to afford the title compound as a white solid in quantitative yield, 3.03 g.

¹H NMR(CDCl₃, 400MHz) δ: 2.19(m, 2H), 2.38(m, 2H), 3.32(m, 2H), 3.45(m, 2H), 5.32(m, 1H), 7.55(d, 1H), 7.78(m, 1H), 8.44(d, 1H), 8.53(m, 1H)

### Preparation 12: N-(4-Chlorophenyl)-4-(pyridin-2-yloxy)piperidine-1-carbothioamide

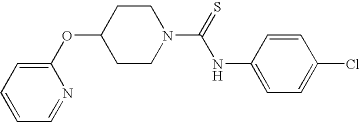

The product of preparation 11 (3.03 g, 11.94 mmol) was partitioned between dichloromethane and saturated sodium hydrogen carbonate solution. The organic phase was then separated, dried over magnesium sulfate and concentrated in vacuo to generate the free base. This intermediate was then dissolved in ethanol (30 mL) and 4-chloro-2-phenylisothiocyanate (1.26 g, 8.02 mmol) was added portionwise and the mixture was stirred for 5 hours. The resulting precipitate was collected by filtration and dried to afford the title compound in 49% yield, 1.36 g.

¹H NMR(DMSO, 400 MHz) δ: 1.71(m, 2H), 2.04(m, 2H), 3.72(m, 2H), 4.24(m, 2H), 5.30(m, 1H), 6.79(d, 1H), 7.30(m, 4H), 7.69(m, 1H), 8.14(d, 1H), 9.38(s, 1H); MS APCI+ m/z 348 [MH]⁺

### Preparation 13: Methyl N-(4-chlorophenyl)-4-(pyridin-2-yloxy)piperidine-1-carbimidothioate

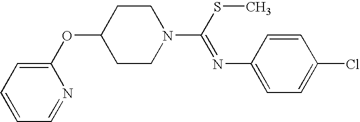

The product of preparation 12 (1.35 g, 3.9 mmol) was stirred in tetrahydrofuran (30 mL), at 0° C. for 10 minutes. Potassium tert-butoxide (480 mg, 4.28 mmol) was added and the solution was stirred at 0° C. for 15 minutes. Methyl tosylate (760 mg, 4.08 mmol) was added and the mixture was stirred at 0° C. for 30 minutes, then at room temperature for 3 hours. The mixture was then diluted with ethyl acetate and washed with brine. The organic solution was dried over magnesium sulfate and concentrated in vacuo to afford the title compound in 91% yield, 1.28 g.

¹H NMR(DMSO, 400 MHz) δ: 1.52(s, 9H), 1.84(m, 4H), 2.23(s, 3H), 2.88(m, 2H), 3.24(m, 2H), 3.52(brs, 1H), 4.41(m, 1H), 7.22(d, 2H), 7.51(d, 2H); MS APCI+ m/z 362 [MH]⁺

### Preparation 14: tert-Butyl (1-{[(4-chlorophenyl)amino]carbonothioyl}-piperidin-4-yl)carbamate

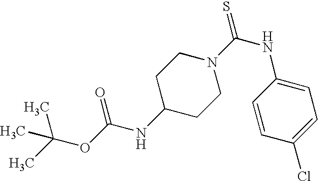

Isothiocyanate (71.3 g, 125.6 mmol) was added to a suspension of 4-(N-BOC amino)piperidine (25.2 g, 125.8 mmol) in ethanol (100 mL) and the mixture was stirred for 15 minutes. The resulting precipitate was collected by filtration and dried to afford the title compound in 91% yield, 42.3 g.

¹H NMR(CDCl₃, 400MHz) δ: 1.29-1.54(m, 11H), 2.01(d, 2H), 3.20(m, 2H), 3.74(brs, 1H), 4.35-4.55(m, 3H), 7.09(d, 2H), 7.17(bs, 1H), 7.32(d, 2H); MS ES+ m/z 392 [MNa]³⁰; Micro analysis found (%); C(55.20), H(6.54), N(11.36); C₁₇H₂₄CIN₃O₂S requires (%); C(55.12), H(6.54), N(11.28).

### Preparation 15: Methyl 4-[(tert-butoxycarbonyl)amino]-N-(4-chlorophenyl)piperidine-1-carbimidothioate

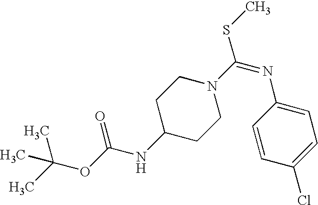

Potassium tert-butoxide (17.8 g, 114.3 mmol) was added to a suspension of the product of preparation 14 (42.3 g, 114.3 mmol) in tetrahydrofuran (200 mL) and the solution was stirred for 10 minutes at room temperature. Methyl tosylate (21.29 g, 114.3 mmol) and additional tetrahydrofuran (200 mL) was then added and the mixture was stirred 10 minutes. Tlc analysis showed that not all of the starting material had been consumed and so further amounts of methyl tosylate (1.08 g, 5.72 mmol) and potassium tert-butoxide (641 mg, 5.72 mmol) were added and the mixture was stirred for 10 minutes. The reaction mixture was then diluted with water (200 mL) and the phases were separated. The organic solution was washed with brine, dried over magnesium sulfate and concentrated in vacuo to afford the title compound as a white solid in quantitative yield.

¹H NMR(CDCl₃, 400MHz) δ: 1.34-1.52(m, 11H), 2.00(d, 2H), 2.05(s, 3H), 3.04(m, 2H), 3.68(brs, 1H), 4.19(d, 2H), 4.50(m, 1H), 6.80(d, 2H), 7.20(d, 2H); MS APCI+ m/z 384 [MH]⁺

### Preparation 16: tert-Butyl {1-[4-(4-chlorophenyl)-5-methyl-4H-1,2,4-triazol-3-yl]piperidin-4-yl}carbamate

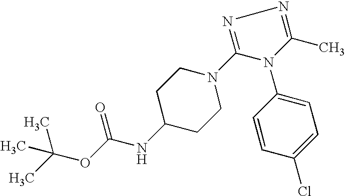

A mixture of the product of preparation 15 (43.89 g, 114.4 mmol), hydrazide (16.9 g, 288 mmol), and trifluoroacetic acid (4 mL, 57.1 mmol) in tetrahydrofuran (250 mL) was heated under reflux for 7 hours. The reaction mixture was then cooled to room temperature and washed with dilute ammonia solution and brine. The aqueous phase was extracted with ethyl acetate and the combined organic extracts were dried over magnesium sulfate and concentrated in vacuo to give an oil. The oil was re-crystallised from ethyl acetate and triturated with diethyl ether to afford the title compound in 72% yield, 32.37 g.

¹H NMR(CD₃OD, 400 MHz) δ: 1.32(m, 2H), 1.40(s, 9H), 1.85(m, 2H), 2.22(s, 3H), 2.84(m, 2H), 3.24(m, 2H), 3.52(bs, 1H), 4.44(m, 1H), 7.24(d, 2H), 7.51(d, 2H); MS APCI⁺ m/z 392 [MH]⁺

### Preparation 17: 1-[4-(4-Chlorophenyl)-5-methyl-4H-1,2,4-triazol-3-yl]-piperidin-4-amine hydrochloride

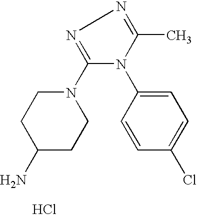

Hydrochloric acid (4M in dioxan, 30 mL) was added to a solution of the product of preparation 16 (32.33 g, 87.5 mmol), in dioxane (200 mL), and the mixture was stirred for 66 hours at room temperature. The reaction mixture was warmed to 50° C. for 3 hours and methanol (50 mL) and additional hydrochloric acid (4M in dioxan, 200 mL) were added at 1.5-hour intervals. The reaction mixture was then concentrated in vacuo and the residue was azeotroped with tetrahydrofuran to afford the title compound as a white solid in quantitative yield, 50 g.

¹H NMR(CD₃OD, 400 MHz) δ: 1.65(m, 2H), 1.96(m, 2H), 2.36(s, 3H), 3.07(m, 2H), 3.36(m, 1H), 3.47(m, 2H), 7.66(d, 2H), 7.75(d, 2H); MS APCI⁺ m/z 292 [MH]⁺

### Preparation 18: tert-Butyl {1-[4-(4-chlorophenyl)-5-methyl-4H-1,2,4-triazol-3-yl]piperidin-4-yl}methylcarbamate

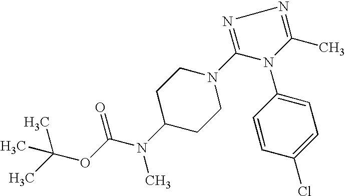

Sodium hydride (60% dispersion in mineral oil, 130 mg, 3.4 mmol) was added to a solution of the product of preparation 16 (250 mg, 0.68 mmol) in tetrahydrofuran (4 mL) and the mixture was stirred for 10 minutes at 5° C. Methyl iodide (84 μL, 1.36 mmol) was then added and the mixture was stirred at room temperature for 2.5 hours. Further methyl iodide (84 μL, 1.36 mmol) was added to the reaction mixture and stirring continued for 18 hours. The solvent was then evaporated under reduced pressure and the residue was partitioned between dichloromethane and water. The aqueous phase was extracted with dichloromethane and the combined organic solutions were dried over magnesium sulfate and concentrated in vacuo. Purification of the residue by column chromatography on silica gel, eluting with dichloromethane:methanol:0.88 ammonia, 95:5:0.5, afforded the title compound in 29% yield, 80 mg.

¹H NMR(CDCl₃, 400MHz) δ: 1.42(s, 9H), 1.50-1.68(m, 4H), 2.22(s, 3H), 2.68(s, 3H), 2.84(m, 2H), 3.33(m, 2H), 4.06(m, 1H), 7.24(d, 2H), 7.50(d, 2H); MS APCI⁺ m/z 406 [MH]⁺

### Preparation 19: 1-[4-(4-Chlorophenyl)-5-methyl-4H-1,2,4-triazol-3-yl]-N-methylpiperidin-4-amine hydrochloride

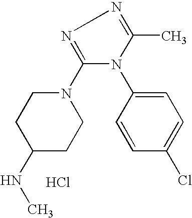

Hydrochloric acid (4M in dioxan, 1 mL) was added to a solution of the product of preparation 18 (80 mg, 0.2 mmol), in methanol (3 mL), and the mixture was stirred for 2 hours at room temperature. The reaction mixture was then concentrated in vacuo to afford the title compound as a white solid in quantitative yield, 90 mg.

¹H NMR(CD₃OD, 400 MHz) δ: 1.69(m, 2H), 2.09(m, 2H), 2.38(s, 3H), 2.69(s, 3H), 3.10(m, 2H), 3.29(m, 1H), 3.50(m, 2H), 7.63-7.82(m, 4H); MS APCI⁺ m/z 306 [MH]⁺

### Preparation 20: N-(1-Benzylpiperidin-4-yl)pyrimidin-2-amine

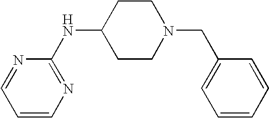

A mixture of 4-amino-1-benzylpiperidine (20.6 g, 108 mmol) and 2-chloropyrimidine (12.4 g, 108 mmol) in ethanol (200 mL) was heated under reflux for 18 hours. The reaction mixture was cooled to room temperature and concentrated in vacuo to low volume until crystallisation was induced. The solid was then filtered off to afford the title compound in 41% yield, 12 g.

¹H NMR(CDCl₃, 400 MHz) δ: 1.57(m, 2H), 2.02(m, 2H), 2.20(m, 2H), 2.81(m, 2H), 3.51(s, 2H), 3.86(m, 1H), 5.00(m, 1H), 6.50(m, 1H), 7.21-7.38(m, 5H), 8.27(m, 2H)

### Preparation 21: N-(1-Benzylpiperidin-4-yl)-N-methylpyridin-2-amine

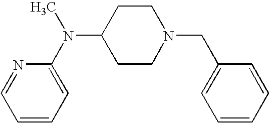

A mixture of 4-methylamino-1-benzylpiperidine [(1.5 g, 6.2 mmol), J. Med. Chem. 39, 3769-89; 1996]1-bromopyridine (0.98 g, 6.2 mmol), sodium tert-butoxide (0.68 g, 7.3 mmol), 1,3-bis(diphenylphosphino)propane (102 mg, 0.24 mmol) and tris(dibenzylideneacetone)dipalladium(0) (113 mg, 0.12 mmol) in toluene (20 mL) was heated under reflux for 4 hours. Tlc analysis showed that there was still starting material present and so further bromopyridine (0.49 g, 3.1 mmol), tris(dibenzylideneacetone)dipalladium(0) (113 mg, 0.12 mmol), bis(diphenylphosphino)propane (102 mg, 0.24 mmol) and sodium tert-butoxide (0.68 g, 7.3 mmol) were added and heating continued for a further 3 hours. The mixture was then diluted with dichloromethane and washed with brine. The organic solution was dried over magnesium sulfate and concentrated in vacuo and the residue was purified by column chromatography on silica gel, eluting with dichloromethane:methanol:0.88 ammonia, 90:10:1 to afford the title compound in 40% yield, 0.69 g.

¹H NMR(CDCl₃, 400 MHz) δ: 1.50-2.40(brm, 6H), 2.65-2.90(m, 5H), 3.55(s, 2H), 4.57(m, 1H), 6.50(m, 2H), 7.24-7.43(m, 6H), 8.18(m, 1H); MS APCI⁺ 282 m/z [MH]⁺

### Preparation 22: N-Piperidin-4-ylpyrimidin-2-amine

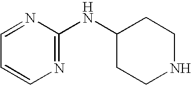

10% Palladium hydroxide (200 mg) was added to a solution of the product of preparation 20 (3 g, 11 mmol), in ethanol (300 mL), and the mixture was stirred at 60° C., under 60 psi of hydrogen, for 2 hours. The reaction mixture was then cooled to room temperature and filtered through Arbocel®, washing through with ethanol. The filtrate was concentrated in vacuo to afford the title product in quantitative yield, 2 g.

¹H NMR(CDCl₃, 400 MHz) δ: 1.38(m, 2H), 2.04(m, 2H), 2.72(m, 2H), 3.06(m, 2H), 3.90(m, 1H), 6.48(m, 1H), 8.26(m, 2H)

### Preparation 23: N-Methyl-N-piperidin-4-ylpyridin-2-amine

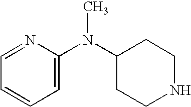

The title compound was prepared from the product of preparation 21, using a similar method to that of preparation 22, as a black oil in 42% yield.

¹H NMR(CDCl₃, 400 MHz) δ: 1.50-2.00(brm, 4H), 2.65-2.90(m, 5H), 3.15(m, 2H), 4.57(m, 1H), 6.50(m, 2H), 7.43(m, 1H), 8.18(m, 1H).

### Preparation 24: N-(4-Chlorophenyl)-4-(pyrimidin-2-ylamino)piperidine-1-carbothioamide

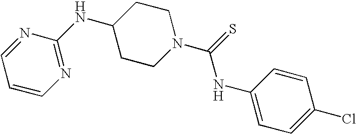

The title compound was prepared from the product of preparation 23 and 4-chlorophenyl isothiocyanate, using a method similar to that of preparation 2, in 72% yield.

¹H NMR(CDCl₃, 400 MHz) δ: 1.60(m, 2H), 2.17(m, 2H), 3.32(m, 2H), 4.11(m, 1H), 4.50(m, 2H), 5.02(m, 1H), 6.56(m, 1H), 7.09(d, 2H), 7.32(d, 2H), 8.09(m, 2H).

### Preparation 25: N-(4-chlorophenyl)-4-[methyl(pyridin-2-yl)amino]piperidine-1-carbothioamide

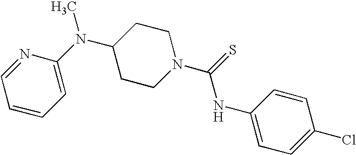

The title compound was prepared from the product of preparation 23 and 4-chlorophenyl isothiocyanate, using a method similar to that of preparation 2, in 49% yield.

¹H NMR(CDCl₃, 400 MHz) δ: 1.78-1.90(m, 4H), 2.89(s, 3H), 3.25(m, 2H), 4.72(m, 2H), 5.03(m, 1H), 6.58(m, 2H), 7.08-7.32(m, 5H), 8.09(m, 1H); MS APCI⁺ 361 m/z [MH]⁺

### Preparation 26: Methyl N-(4-chlorophenyl)-4-(pyrimidin-2-ylamino)piperidine-1-carbimidothioate

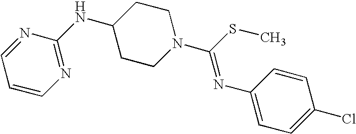

The title compound was prepared from the product of preparation 24, using a method similar to that of preparation 4, in 35% yield.

¹H NMR(CDCl₃, 400 MHz) δ: 1.52(m, 2H), 2.04(2xs, isomers, 3H), 2.12(m, 2H), 3.13(m, 2H), 4.25(m, 2H), 5.09(m, 1H), 6.55(m, 1H), 6.82(d, 2H), 7.20(d, 2H), 8.29(m, 2H); MS APCI⁺ 362 m/z [MH]⁺

### Preparation 27: Methyl N-(4-chlorophenyl)-4-[methyl(pyridin-2-yl)amino]piperidine-1-carbimidothioate

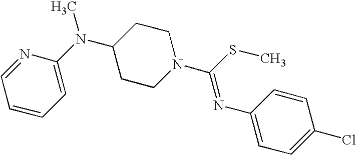

The title compound was prepared from the product of preparation 25, using a similar method to preparation 4, in 92% yield.

¹H NMR(CDCl₃, 400 MHz) δ: 1.78(m, 4H), 2.07(s, 3H), 2.95(s, 3H), 3.03(m, 2H), 4.42(m, 2H), 4.86(m, 1H), 6.50(d, 1H), 6.55(m, 1H), 6.82(d, 2H), 7.20(d, 2H), 7.46(m, 1H), 8.18(m, 1H); MS APCI⁺ 375 m/z [MH]⁺

### Preparation 28: tert-Butyl 4-(pyrimidin-2-yloxy)piperidine-1-carboxylate

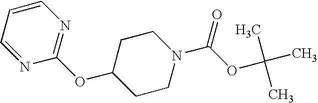

1-tert-Butoxycarbonyl-4-hydroxypiperidine (10 g, 50 mmol) was dissolved in tetrahydrofuran (200 mL) and cooled to 5° C. Sodium hydride (60% dispersion in mineral oil, 2.5 g, 65 mmol) was added portionwise and the resulting mixture was stirred for 30 minutes at 5° C. 1-Chloropyrimidine (11.4 g, 99 mmol) was added and the mixture was stirred for 18 hours at room temperature. The mixture was then filtered, concentrated in vacuo and the residue was partitioned between ethyl acetate and water. The organic layer was separated, dried over magnesium sulfate and concentrated in vacuo. Re-crystallisation of the residue for diisopropyl ether (50 mL) afforded the title compound in 68% yield, 9.5 g.

¹H NMR(CD₃OD, 400 MHz) δ: 1.49(s, 9H), 1.76(m, 2H), 2.03(m, 2H), 3.36(m, 2H), 3.78(m, 2H), 5.28(m, 1H), 7.09(m, 1H), 8.57(m, 2H); MS ES⁺ 280 m/z [MH]⁺

### Preparation 29: 2-(Piperidin-4-yloxy)pyrimidine hydrochloride

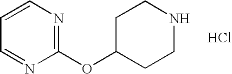

The title compound was prepared from the product of preparation 28, using a method similar to that of preparation 11, as a solid in quantitative yield, 8.5 g.

¹H NMR(CD₃OD, 400 MHz) δ: 2.20(m, 2H), 2.35(m, 2H), 3.32(m, 2H), 3.43(m, 2H), 5.58(m, 1H), 7.42(m, 1H), 8.86(m, 2H); MS ES⁺ 180 m/z [MH]⁺

### Preparation 30: N-(4-Chlorophenyl)-4-(pyrimidin-2-yloxy)piperidine-1-carbothioamide

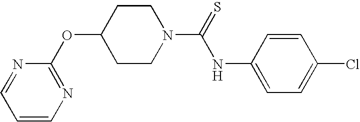

The title compound was prepared from the product of preparation 29, using a method similar to that of preparation 2. The crude compound was re-crystallised from ethyl acetate and diisopropyl ether to afford the title compound in 79% yield.

¹H NMR(CD₃OD, 400 MHz) δ: 1.91(m, 2H), 2.15(m, 2H), 3.95(m, 2H), 4.22(m, 2H), 5.39(m, 1H), 7.11 (d, 1H), 7.30(m, 4H), 8.58(m, 2H); MS APCI⁺ m/z 349 [MH]⁺

### Preparation 31: Methyl N-(4-chlorophenyl)-4-(pyrimidin-2-yloxy)piperidine-1-carbimidothioate

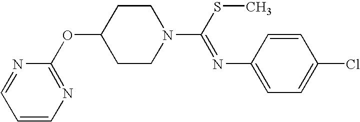

The title compound was prepared from the product of preparation 30, using a method similar to that of preparation 4, in quantitative yield, 9.9 g.

¹H NMR(CD₃OD, 400 MHz) δ: 1.90(m, 2H), 2.15(m, 5H), 3.53(m, 2H), 3.97(m, 2H), 5.32 (m, 1H), 6.82(d, 2H), 7.09(m, 1H), 7.22(d, 2H), 8.59(m, 2H); MS APCI⁺ 363 m/z [MH]⁺

### Preparation 32: Methyl 2H-1,2,3-triazol-2-ylacetate

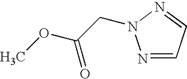

A mixture of 1,2,3-triazole (10 g, 144 mmol), methyl bromoacetate (22 g, 144 mmol) and potassium carbonate (20 g, 144 mmol) in acetonitrile (100 mL) was heated under reflux for 18 hours. The mixture was then diluted with ethyl acetate, washed with water and brine, dried over magnesium sulfate and concentrated in vacuo to afford the title compound in 56% yield, 11.4 g.

¹H NMR(CDCl₃, 400 MHz) δ: 3.79(2xs, isomers, 3H), 5.22(2xs, isomers, 2H), 7.70(d, 1H), 7.89(d, 1H)

### Preparation 33: 2-(2H-1,2,3-Triazol-2-yl)acetohydrazide

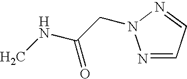

A mixture of the product of preparation 32 (1 g, 7.1 mmol) and hydrazine monohydrate (1.4 g, 29 mmol) in methanol (6 mL) was heated under reflux for 24 hours. The reaction mixture was then cooled to room temperature and was evaporated under reduced pressure to give an oily residue. The residue was re-crystallised from methanol to afford the title compound as a crystalline solid in 24% yield, 245 mg.

¹H NMR(CDCl₃, 400 MHz) δ: 3.90(brs, 2H), 5.22(2xs, isomers, 2H), 7.29(s, 1H), 7.80(s, 1H)

### Preparation 34: 3-(3-Chloro-phenoxy)-azetidine-1-carbothioic acid (4-chloro-phenyl)-amide

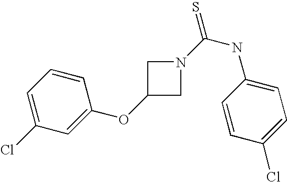

2.0 g (10.9 mmoles, 1 eq.) of 3-(3-chloro-phenoxy)-azetidine were dissolved in 100 ml of EtOH and 1.9 g (11.4 mmoles, 1.05 eq.) of 1-chloro-4-isothiocyanato-benzene added. The solution was stirred at room temperature overnight. The solvent was removed under reduced pressure, affording a white solid. This solid was purified by column chromatography (using a gradient of DCM and pentane) affording 750 mg of the title compound (19.5%) as a white solid. ¹HNMR(CDCl₃, 400 MHz): 4.20(s, 2H), 4.42(m, 2H), 4.60 (m, 1H), 5.10 (s, 1H), 6.60 (m, 1H), 6.70 (m, 1H), 7.00 (m, 1H), 7.05-7.40(m, 5H); MS ES+ m/z 317 [MH]⁺355

### Preparation 36: 3-(3-Chloro-phenoxy)-N-(4-chloro-phenyl)-azetidine-1-carboximidothioic acid methyl ester

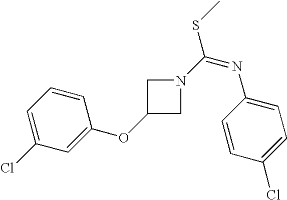

750 mg (2.1 mmoles, 1 eq.) of the compound from preparation 34 were dissolved in 50 ml of THF, and then 238 mg (2.1 mmoles, 1 eq.) of potassium tert-butoxide were added. The solution was stirred for 5 minutes at room temperature, and then 395 mg (2.1 mmoles, 1 eq.) of methyl tosylate were added. The mixture was stirred over night, the solvent was removed under reduced pressure. The residue was then taken up in AcOEt, the solution was washed with water, followed by brine, and then dried over MgSO₄. The volatiles were removed under reduced pressure, affording 810 mg of the title compound (>100%), which was used directly in the preparation of Example 51.

### EXAMPLE 1

### 1-[4-(4-Chloro-2-methylphenyl)-5-methyl-4H-1,2,4-triazol-3-yl]-4-(3-chlorophenoxy)piperidine

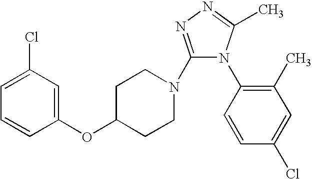

Polymer supported triphenyl phosphine (300mg), di-tert-butyl azodicarboxylate (157 mg, 0.68 mmol) and the product of preparation 8 (100 mg, 0.34 mmol) were added to an ice-cold solution of 3-chlorophenol (87 mg, 0.68 mmol) in dichloromethane (2 mL) and the reaction mixture was stirred at room temperature for 3 hours. The reaction mixture was then filtered through a filter tube, washing through with dichloromethane. Trifluoroacetic acid (1 mL) was added to the filtrate and the mixture was stirred for 45 minutes. The reaction mixture was washed with 6M sodium hydroxide solution and the organic layer was dried over magnesium sulfate and concentrated in vacuo. The residue was purified by HPLC using a Phenomenex Luna C18 system, eluting with 5:95 to 95:5 acetonitrile:acetonitrile/diethylamine (99.9:0.1), to afford the title compound in 13% yield, 18 mg.

¹H NMR(CD₃OD, 400 MHz) δ: 1.60(m, 2H), 1.81(m, 2H), 2.02(s, 3H), 2.06(s, 3H), 2.97(m, 2H), 3.13-3.43(brm, 2H), 4.42(m, 1H), 6.78(d, 1H), 6.83(m, 2H), 7.12(m, 1H), 7.32(d, 1 H), 7.39(d, 1H), 7.49(s, 1H); MS ES⁺ m/z 417, 423 [MH]⁺

### EXAMPLES 2 TO 30

The following compounds of the general formula shown below were prepared from preparations 8 and 9, using a similar method to example 1.

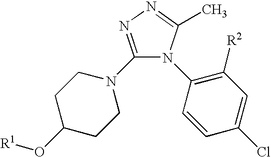

| Ex. | | | | |
|---|---|---|---|---|
| No. | R¹ | R² | Data | Yield |
| 2 | 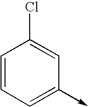 | H | ¹H NMR(CD₃OD, 400MHz) δ: 1.61(m, 2H), 1.82(m, 2H), 2.16(s, 3H), 2.93(m, 2H), 3.13- 3.43(brm, 2H), 4.43(m, 1H), 6.78(d, 1H), 6.85(m, 2H), 7.14(m, 1H), 7.42(d, 2H), 7.58(d, 2H); MS ES+ m/z 403 [MH]⁺ | 15% |
| 3 | 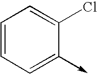 | CH₃ | ¹HNMR(CD₃OD, 400MHz) δ: 1.67(m, 2H), 1.79(m, 2H), 2.02(s, 3H), 2.06(s, 3H), 2.97(m, 2H), 3.13-3.43(brm, 2H), 4.58(m, 1H), 6.84(d, 1H), 7.00(d, 1H), 7.18(m, 1H), 7.27(d, 1H), 7.31(d, 1H), 7.39(d, 1H), 7.50(s, 1H); MS ES+ m/z 417 [MH]⁺ | 42% |
| 4 | 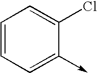 | H | ¹H NMR(CD₃OD, 400MHz) δ: 1.67(m, 2H), 1.82(m, 2H), 2.17(s, 3H), 2.95(m, 2H), 3.17- 3.42(brm, 2H), 4.58(m, 1H), 6.84(d, 1H), 7.01(d, 1H), 7.18(m, 1H), 7.29(d, 1H), 7.43(d, 2H), 7.58(d, 2H); MS ES+ m/z 403 [MH]⁺ | 15% |
| 5 | 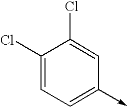 | CH₃ | ¹H NMR(CD₃OD, 400MHz) δ: 1.39(m, 2H), 1.80(m, 2H), 2.02(s, 3H), 2.06(s, 3H), 2.98(m, 2H), 3.13-3.43(brm, 2H), 4.44(m, 1H), 6.80(d, 1H), 7.04(d, 1H), 7.32(m, 2H), 7.40(d, 1H), 7.48(s, 1H); MS ES+ m/z 451 [MH]⁺ | 18% |
| 6 | 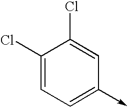 | H | ¹H NMR(CD₃OD, 400MHz) δ: 1.61(m, 2H), 1.84(m, 2H), 2.18(s, 3H), 2.94(m, 2H), 3.15- 3.45(brm, 2H), 4.42(m, 1H), 6.81(d, 1H), 7.03(s, 1H), 7.36(d, 1H), 7.42(d, 2H), 7.58(d, 2H); MS ES+ m/z 439 [MH]⁺ | 15% |
| 7 | 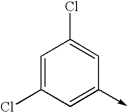 | CH₃ | ¹H NMR(CD₃OD, 400MHz) δ: 1.59(m, 2H), 1.80(m, 2H), 2.02(s, 3H), 2.06(s, 3H), 2.95(m, 2H), 3.13-3.43(brm, 2H), 4.46(m, 1H), 6.84(s, 2H), 6.90(s, 1H), 7.32(d, 1H), 7.40(d, 1H), 7.49(s, 1H); MS ES+ m/z 451, 457 [MH]⁺ | 17% |
| 8 | 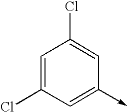 | H | ¹H NMR(CD₃OD, 400MHz) δ: 1.60(m, 2H), 1.83(m, 2H), 2.16(s, 3H), 2.94(m, 2H), 3.13- 3.43(brm, 2H), 4.45(m, 1H), 6.84(s, 2H), 6.92(s, 1H), 7.42(d, 2H), 7.58(d, 2H); MS ES+ m/z 439 [MH]⁺ | 17% |
| 9 | 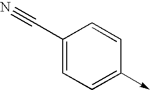 | CH₃ | ¹H NMR(CD₃OD, 400MHz) δ: 1.61(m, 2H), 1.83(m, 2H), 2.02(s, 3H), 2.06(s, 3H), 2.98(m, 2H), 3.13-3.43(brm, 2H), 4.59(m, 1H), 6.99(d, 2H), 7.31(d, 1H), 7.40(d, 1H), 7.49(s, 1H), 7.55(d, 2H); MS ES+ m/z 408 [MH]⁺ | 12% |
| 10 | 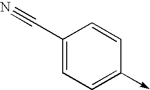 | H | ¹HNMR(CD₃OD, 400MHz) δ: 1.64(m, 2H), 1.88(m, 2H), 217(s, 3H), 2.95(m, 2H), 3.14- 3.43(brm, 2H), 4.59(m, 1H), 7.00(d, 2H), 7.45(d, 2H), 7.59(m, 4H); MS ES+ m/z 394 [MH]⁺ | 14% |
| 11 | 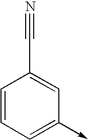 | CH₃ | ¹H NMR(CD₃OD, 400MHz) δ: 1.60(m, 2H), 1.83(m, 2H), 2.02(s, 3H), 2.06(s, 3H), 2.98(m, 2H), 3.13-3.43(brm, 2H), 4.59(m, 1H), 7.20(m, 3H), 7.30-7.42(m, 3H), 7.50(s, 1H); MS ES+ m/z 408 [MH]⁺ | 17% |
| 12 | 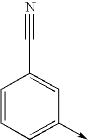 | H | ¹H NMR(CD₃OD, 400MHz) δ: 1.60(m, 2H), 1.82(m, 2H), 2.12(s, 3H), 2.91(m, 2H), 3.10- 3.42(brm, 2H), 4.59(m, 1H), 7.18(m, 4H), 7.22(m, 2H), 7.53(m, 2H); MS ES+ m/z 394 [MH]⁺ | 11% |
| 13 | 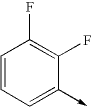 | CH₃ | ¹H NMR(CD₃OD, 400MHz) δ: 1.63(m, 2H), 1.82(m, 2H), 2.02(s, 3H), 2.06(s, 3H), 2.96(m, 2H), 3.13-3.43(brm, 2H), 4.45(m, 1H), 6.73(m, 1H), 6.85(m, 1H), 6.99(m, 1H), 7.32(d, 1H), 7.40(d, 1H), 7.50(s, 1H); MS ES+ m/z 419 [MH]⁺ | 18% |
| 14 | 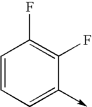 | H | ¹H NMR(CD₃OD, 400MHz) δ: 1.65(m, 2H), 1.84(m, 2H), 2.15(s, 3H), 2.94(m, 2H), 3.13- 3.43(brm, 2H), 4.45(m, 1H), 6.77(m, 1H), 6.85(m, 1H), 6.95(m, 1H), 7.42(d, 2H), 7.58(d, 2H); MS ES+ m/z 405 [MH]⁺ | 14% |
| 15 | 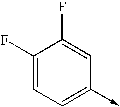 | CH₃ | ¹H NMR(CD₃OD, 400MHz) δ: 1.59(m, 2H), 1.80(m, 2H), 2.02(s, 3H), 2.06(s, 3H), 2.89- 3.01(m, 2H), 3.13-3.43(brm, 2H), 4.39(m, 1H), 6.64(m, 1H), 6.80(m, 1H), 6.99(m, 1H), 7.05(m, 1H), 7.30(d, 1H), 7.40(d, 1H), 7.50(s, 1H); MS ES+ m/z 419 [MH]⁺ | 17% |
| 16 | 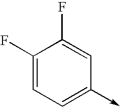 | H | ¹H NMR(CD₃OD, 400MHz) δ: 1.59(m, 2H), 1.82(m, 2H), 2.15(s, 3H), 2.93(m, 2H), 3.15- 3.45(brm, 2H), 4.38(m, 1H), 6.62(m, 1H), 6.79(m, 1H), 7.05(m, 1H), 7.43(d, 2H), 7.58(d, 2H); MS ES+ m/z 405 [MH]⁺ | 17% |
| 17 | 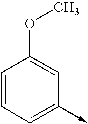 | CH₃ | ¹H NMR(CD₃OD, 400MHz) δ: 1.58(m, 2H), 1.78(m, 2H), 2.02(s, 3H), 2.06(s, 3H), 2.90-3.01(m, 2H), 3.13-3.43(brm, 2H), 3.68(s, 3H), 4.39(m, 1H), 6.35(m, 1H), 6.42(d, 2H), 7.06(m, 1H), 7.30(d, 1H), 7.40(d, 1H), 7.48(s, 1H); MS ES+ m/z 413 [MH]⁺ | 23% |
| 18 | 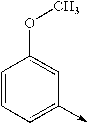 | H | ¹H NMR(CD₃OD, 400MHz) δ: 1.61(m, 2H), 1.82(m, 2H), 2.18(s, 3H), 2.92(m, 2H), 3.14- 3.43(brm, 2H), 3.66(s, 3H), 4.40(m, 1H), 6.42(m, 3H), 7.05(m, 1H), 7.42(d, 2H), 7.56(d, 2H); MS ES+ m/z 399 [MH]⁺ | 12% |
| 19 | 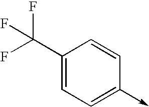 | CH₃ | ¹H NMR(CD₃OD, 400MHz) δ: 1.62(m, 2H), 1.83(m, 2H), 2.02(s, 3H), 2.05(s, 3H), 2.91- 3.02(m, 2H), 3.13-3.42(brm, 2H), 4.58(m, 1H), 6.99(m, 2H), 7.30(d, 1H), 7.40(d, 1H), 7.45(m, 3H); MS ES+ m/z 451 [MH]⁺ | 21% |
| 20 | 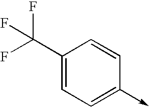 | H | ¹H NMR(CD₃OD, 400MHz) δ: 1.63(m, 2H), 1.88(m, 2H), 2.17(s, 3H), 2.97(m, 2H), 3.15- 3.42(brm, 2H), 4.58(m, 1H), 6.99(m, 2H), 7.40-7.51(m, 4H), 7.60(d, 2H); MS ES+m/z 437 [MH]⁺ | 17% |
| 21 | 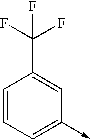 | CH₃ | ¹H NMR(CD₃OD, 400MHz) δ: 1.61(m, 2H), 1.82(m, 2H), 2.02(s, 3H), 2.04(s, 3H), 2.91- 3.02(m, 2H), 3.13-3.42(brm, 2H), 4.58(m, 1H), 7.10(m, 3H), 7.36(d, 1H), 7.38(m, 2H), 7.49(d, 1H); MS ES+ m/z 451 [MH]⁺ | 20% |
| 22 | 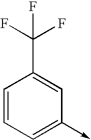 | H | ¹H NMR(CD₃OD, 400MHz) δ: 1.64(m, 2H), 1.85(m, 2H), 2.17(s, 3H), 2.95(m, 2H), 3.15- 3.43(brm, 2H), 4.58(m, 1H), 7.10(m, 3H), 7.38(m, 1H), 7.42(d, 2H), 7.58(d, 2H); MS ES+ m/z 437 [MH]⁺ | 15% |
| 23 | 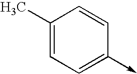 | CH₃ | ¹H NMR(CD₃OD, 400MHz) δ: 1.55(m, 2H), 1.78(m, 2H), 2.02(s, 3H), 2.04(s, 3H), 2.16(s, 3H), 2.86-3.00(m, 2H), 3.12- 3.43(brm, 2H), 4.32(m, 1H), 6.70(d, 2H), 6.98(d, 2H), 7.30(d, 1H), 7.39(d, 1H) 7.48(d, 1H); MS ES+ m/z 397 [MH]⁺ | 24% |
| 24 | 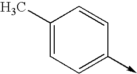 | H | ¹H NMR(CD₃OD, 400MHz) δ: 1.58(m, 2H), 1.79(m, 2H), 2.17(m, 6H), 2.90(m, 2H), 3.13-3.43(brm, 2H), 4.34(m, 1H), 6.72(d, 2H), 6.99(d, 2H), 7.42(d, 2H), 7.58(d, 2H); MS ES+ m/z 383 [MH]⁺ | 20% |
| 25 | 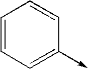 | CH₃ | ¹H NMR(CD₃OD, 400MHz) δ: 1.58(m, 2H), 1.80(m, 2H), 2.02(s, 3H), 2.06(s, 3H), 2.86- 3.00(m, 2H), 3.11-3.41(brm, 2H), 4.39(m, 1H), 6.82(m, 3H), 7.18(d, 2H), 7.31(d, 1H), 7.39(d, 1H) 7.49(d, 1H); MS ES+ m/z 383 [MH]⁺ | 9% |
| 26 | 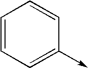 | H | ¹H NMR(CD₃OD, 400MHz) δ: 1.59(m, 2H), 1.82(m, 2H), 2.14(s, 3H), 2.92(m, 2H), 3.15- 3.43(brm, 2H), 4.39(m, 1H), 6.82(m, 3H), 7.18(m, 2H), 7.42(d, 2H), 7.57(d, 2H); MS ES+ m/z 369 [MH]⁺ | 18% |
| 27 | 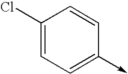 | CH₃ | ¹H NMR(CD₃OD, 400MHz) δ: 1.58(m, 2H), 1.79(m, 2H), 2.02(s, 3H), 2.04(s, 3H), 2.89- 3.01(m, 2H), 3.11-3.43(brm, 2H), 4.38(m, 1H), 6.82(d, 2H), 7.14(d, 2H), 7.30(d, 1H), 7.40(d, 1H) 7.45(d, 1H); MS ES+ m/z 417 [MH]⁺ | 11% |
| 28 | 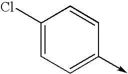 | H | ¹H NMR(CD₃OD, 400MHz) δ: 1.61(m, 2H), 1.81(m, 2H), 2.15(s, 3H), 2.91(m, 2H), 3.15- 3.43(brm, 2H), 4.40(m, 1H), 6.84(d, 2H), 7.18(d, 2H), 7.44(d, 2H), 7.58(d, 2H); MS ES+ m/z 403 [MH]⁺ | 18% |
| 29 | 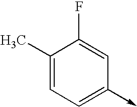 | CH₃ | ¹H NMR(CD₃OD, 400MHz) δ: 1.57(m, 2H), 1.79(m, 2H), 2.02(s, 3H), 2.05(s, 3H), 2.10(s, 3H), 2.89-3.02(m, 2H), 3.14- 3.42(brm, 2H), 4.37(m, 1H), 6.58(m, 2H), 7.00(m, 1H), 7.30(d, 1H), 7.40(d, 1H) 7.48(s, 1H); MS ES+ m/z 415 [MH]⁺ | 11% |
| 30 | 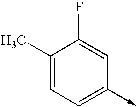 | H | ¹H NMR(CD₃OD, 400MHz) δ: 1.68(m, 2H), 1.81(m, 2H), 2.04(s, 3H), 2.15(s, 3H), 2.91(m, 2H), 3.12-3.42(brm, 2H), 4.37(m, 1H), 6.54(m, 2H), 6.99(m, 1H), 7.42(d, 2H), 7.58(d, 2H); MS ES+ m/z 401 [MH]⁺ | 18% |

### EXAMPLE 31

### 2-({1-[4-(4-Chlorophenyl)-5-methyl-4H-1,2,4-triazol-3-yl]piperidine-4-yl}oxy)pyridine

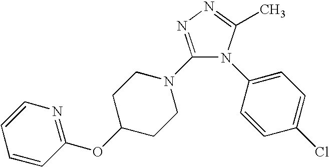

A mixture of the product of preparation 9 (100 mg, 0.34 mmol), sodium hydride (60% dispersion in mineral oil, 30 mg, 0.68 mmol), 1-methyl-2-pyrrolidinone (2 mL), and 2-chloropyridine (78 μL, 0.68 mmol) were heated under reflux for 18 hours. The reaction mixture was then diluted with ethyl acetate and washed with brine (×5). The organic solution was dried over magnesium sulfate and concentrated in vacuo. Purification of the residue by column chromatography on silica gel, eluting with dichloromethane:methanol:0.88 ammonia 90:10:1, afforded the title compound as a foam in 54% yield, 69 mg.

¹H NMR(CDCl₃, 400MHz) δ: 1.68(m, 2H), 1.95(m, 2H), 2.21(s, 3H), 2.96(m, 2H), 3.26(m, 2H), 5.11(m, 1H), 6.63(m, 1H), 6.80(m, 1H), 7.28(m, 2H), 7.52(m, 3H), 8.05(d, 1H); MS APCI+ m/z 370 [MH]⁺

### EXAMPLE 32

### 2-({1-[4-(4-Chlorophenyl)-5-methyl-4H-1,2,4-triazol-3-yl]-piperidin-4-yl}oxy)pyrimidine

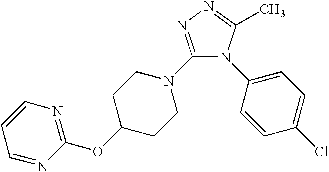

Sodium hydride (60% dispersion in mineral oil, 17 mg, 0.44 mmol) was added to an ice-cold solution of the product of preparation 9 (100 mg, 0.34 mmol) in tetrahydrofuran (2 mL) and the mixture was stirred for 20 minutes. 2-Chloropyrimidine (78 mg, 0.68 mmol) was added and the mixture was stirred at room temperature for 3 hours. The reaction mixture was then diluted with dichloromethane, washed with brine, dried over magnesium sulfate and concentrated in vacuo. The residue was purified by column chromatography on silica gel, eluting with dichloromethane:methanol:0.88 ammonia, 90:10:1, to afford the title compound in 48% yield, 60.7 mg.

¹H NMR(CDCl₃, 400 MHz) δ: 1.73(m, 2H), 1.99(m, 2H), 2.22(s, 3H), 2.98(m, 2H), 3.32(m, 2H), 5.06(m, 1H), 6.89(m, 1H), 7.27(m, 2H), 7.45(m, 2H), 8.45(d, 2H); MS APCI+ m/z 371 [MH]⁺

### EXAMPLE 33

### 2-({1-[4-(4-Chlorophenyl)-5-(methoxymethyl)-4H-1,2,4-triazol-3-yl]piperidin-4-yl}oxy)pyridine

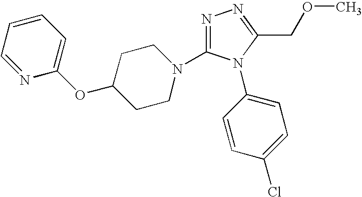

A mixture of the product of preparation 13 (252 g, 0.69 mmol) methoxyacethydrazide (80 mg, 0.77 mmol) and trifluoroacetic acid (26 μL, 0.35 mmol) in tetrahydrofuran (15 mL) was heated under reflux for 5 hours. The reaction mixture was then cooled to room temperature, diluted with ethyl acetate and washed with brine. The organic phase was dried over magnesium sulfate and concentrated in vacuo and the residue was purified by column chromatography on silica gel, eluting with dichloromethane:methanol: 95:5, to afford the title compound in 65% yield, 180 mg.

¹H NMR(CD₃OD, 400 MHz) δ: 1.73(m, 2H), 1.98(m, 2H), 3.05(m, 2H), 3.37(m, 5H), 4.31(s, 2H), 5.16(m, 1H), 6.68(m, 1H), 6.82(m, 1H), 7.42-7.58(m, 5H), 8.09(d, 1H); MS ES+ m/z 400 [MH]⁺

### EXAMPLE 34 TO 38

The following compounds of general formula shown below were prepared from the product of preparation 31 and the appropriate hydrazide, using a similar method to example 33:

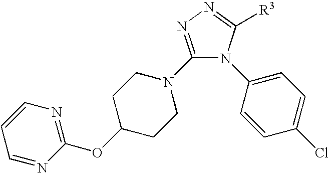

| No. | R³ | Data | Yield |
|---|---|---|---|
| 34 | 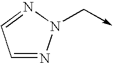 | ¹H NMR(CDCl₃, 400MHz) δ: 1.73(m, 2H), 1.98(m, 2H), 3.02(m, 2H), 3.35(m, 2H), 5.06(m, 1H), 5.58(s, 2H), 6.89(m, 1H), 7.12(d, 2H), 7.38(d, 2H), 7.49(s, 2H), 8.45(d, 2H); MS APCI⁺ 438 m/z [MH]⁺ | 23% |
| 35 | 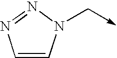 | ¹H NMR(CD₃OD, 400MHz) δ: 1.73(m, 2H), 1.98 (m, 2H), 3.06(m, 2H), 3.35(m, 2H), 5.09(m, 1H), 5.68(s, 2H), 7.05(m, 1H), 7.39(d, 2H), 7.59(d, 2H), 7.62(s, 1H), 7.81(s, 1H), 8.55(d, 2H); MS APCI⁺ 438 m/z m/z [MH]⁺ | |
| 36 | 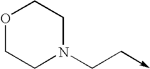 | ¹H NMR(CDCl₃, 400MHz) δ: 1.72(m, 2H), 1.99(m, 2H), 2.34(m, 4H), 2.68(m, 4H), 2.97(m, 2H), 3.30(m, 2H), 3.60(m, 4H), 5.08(m, 1H), 6.89(m, 1H), 7.29(m, 2H), 7.56(d, 2H), 8.47(d, 2H); MS APCI⁺ 470 m/z [MH]⁺ | 68% |
| 37 | 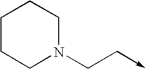 | ¹H NMR(CDCl₃, 400MHz) δ: 1.39(m, 2H), 1.50(m, 4H), 1.76(m, 2H), 2.00(m, 2H), 2.30(m, 4H), 2.69(m, 4H), 2.99(m, 2H), 3.32(m, 2H), 5.09(m, 1H), 6.90(m, 1H), 7.29(m, 2H), 7.50(d, 2H), 8.50(d, 2H); MS APCI⁺ 468 m/z [MH]⁺ | 40% |
| 38 | 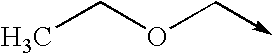 | ¹H NMR(CD₃OD, 400MHz) δ: 1.18(t, 3H), 1.78(m, 2H), 2.00(m, 2H), 3.05(m, 2H), 3.27-3.45(m, 4H), 4.39(s, 2H), 5.20(m, 1H), 7.05(m, 1H), 7.56(d, 2H), 7.62(d, 2H), 8.55(d, 2H); MS APCI+ m/z 415 [MH]⁺ | 23% |

### EXAMPLES 36 AND 37

### Hydrazide Intermediates Can Be Purchased From Chem. Div. Inc.

### EXAMPLE 39

### N-{1-[4-(4-Chlorophenyl)-5-methyl-4H-1,2,4-triazol-3-yl]-piperidin-4-yl}pyrimidin-2-amine

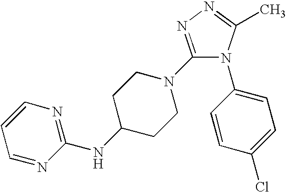

The title compound was prepared from the product of preparation 26 and acethydrazide, using a similar method to that of example 33, as a solid in 58% yield.

¹H NMR(CDCl₃, 400 MHz) δ: 1.44(m, 2H), 2.00(m, 2H), 2.23(s, 3H), 2.96(m, 2H), 3.31(m, 2H), 3.94(m, 1H), 4.96(m, 1H), 6.51(m, 1H), 7.28(d, 2H), 7.52(d, 2H), 8.24(m, 2H); MS APCI⁺ 370 m/z [MH]⁺

### EXAMPLE 40

### N-{1-[4-(4-Chlorophenyl)-5-methyl-4H-1,2,4-triazol-3-yl]-piperidin-4-yl}-N-methylpyridin-2-amine

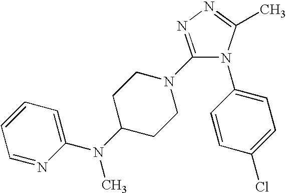

The title compound was prepared from the product of preparation 27 and acethydrazide, using a similar method to that of example 33, as a solid in 58% yield.

¹H NMR(CDCl₃, 400MHz) δ: 1.56-1.78(m, 4H), 2.22(s, 3H), 2.80(s, 3H), 2.98(m, 2H), 3.38(m, 2H), 4.63(m, 1H), 6.44(d, 1H), 6.51(m, 1H), 7.25(d, 2H), 7.42(m, 1H), 7.50(d, 2H), 8.10(m, 1H); MS APCI⁺ 383 m/z [MH]⁺

### EXAMPLE 41

### N-{1-[4-(4-Chlorophenyl)-5-methyl-4H-1,2,4-triazol-3-yl]-piperidin-4-yl}-N-methylpyrimidin-2-amine

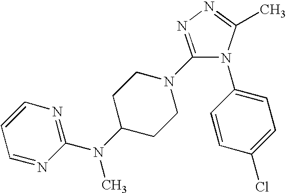

A mixture of the product of preparation 19 (90 mg, 0.2 mmol), 2-chloropyrimidine (23 mg, 0.2 mmol) and N,N-diisopropylethylamine (130 mg, 1 mmol) in ethanol (2 mL) was heated under reflux for 18 hours. Tlc analysis showed that 50% starting material remained and so 2-bromopyrimidine (32 mg, 0.2 mmol), potassium carbonate (27.6 mg, 0.4 mmol) sodium iodide (10 mg) and 1-methyl-2-pyrrolidinone (2 mL) were added and the reaction mixture was heated under reflux for a further 18 hours. The mixture was then diluted with ethyl acetate, washed with brine, dried over magnesium sulfate and concentrated in vacuo. Purification of the residue by column chromatography on silica gel, eluting with dichloromethane:methanol:0.88 ammonia, 90:10:1, afforded the title compound in 30% yield, 23 mg.

¹H NMR(CD₃OD, 400 MHz) δ: 1.58(m, 2H), 1.77(m, 2H), 2.23(s, 3H), 2.82-3.00(m, 5H), 3.38(m, 2H), 4.70(m, 1H), 6.57(m, 1H), 7.50(d, 2H), 7.65(d, 2H), 8.30(m, 2H); MS APCI⁺ 384 m/z [MH]⁺

### EXAMPLE 42

### N-{1-[4-(4-Chlorophenyl)-5-methyl-4H-1,2,4-triazol-3-yl]-piperidin-4-yl}pyridin-2-amine

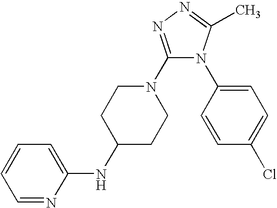

A mixture of the product of preparation 17 (100 mg, 0.34 mmol), 1-bromopyridine (54 mg, 0.34 mmol), sodium tert-butoxide (38 mg, 0.39 mmol), 1,3-bis(diphenylphosphino)propane (5.6 mg, 14 μmol) and tris(dibenzylideneacetone)dipalladium(0) (6.3 mg, 6.8 μmol) in toluene (5 mL) was heated under reflux for 18 hours. The mixture was then diluted with ethyl acetate and washed with brine and the organic solution was dried over magnesium sulfate and concentrated in vacuo. The residue was purified by column chromatography on silica gel, eluting with dichloromethane:methanol:0.88 ammonia, 90:10:1 and the relevant fraction was triturated with ether to afford the title compound in 6% yield, 8 mg.

¹H NMR(CD₃OD, 400 MHz) δ: 1.39(m, 2H), 1.89(m, 2H), 2.20(s, 3H), 2.92(m, 2), 3.18-3.38(m, 2H), 3.75(m, 1H), 6.50(m, 2H), 7.39(d, 1H), 7.51(d, 2H), 7.64(m, 2H), 7.89(m, 1H); MS APCI⁺ 370 m/z [MH]⁺

### EXAMPLE 43

### N-{1-[4-(4-Chlorophenyl)-5-methyl-4H-1,2,4-triazol-3-yl]-piperidin-4-yl}-3-nitropyridin-2-amine

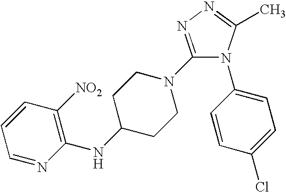

Triethylamine (0.84 mL, 6.03 mmol) and 2-chloro-3-nitropyridine (319 mg, 2.01 mmol) were added to a suspension of the product of preparation 17 (1 g, 2.01 mmol) in tetrahydrofuran (10 mL) and the mixture was stirred for 18 hours at room temperature. N,N-Dimethylformamide (3 drops) was added and the mixture was heated under reflux for 24 hours. The solvent was then evaporated under reduced pressure and the residue was partitioned between water and dichloromethane. The organic layer was separated, washed with brine, dried over magnesium sulfate and concentrated in vacuo. The residue was purified by column chromatography on silica gel, eluting with dichloromethane:methanol:0.88 ammonia, 100:0:0 to 95:5:0.5. The appropriate fractions were evaporated under reduced pressure and the residue was azeotroped with ethyl acetate to afford the title compound as a yellow solid in 30% yield, 250 mg.

¹H NMR(CDCl₃, 400 MHz) δ: 1.47(m, 2H), 1.97(m, 2H), 2.18(s, 3H), 2.93(m, 2H), 3.29(m, 2H), 4.24(m, 1H), 6.56(d, 1H), 7.30(d, 2H), 7.54(d, 2H), 8.05(d, 1H), 8.36(m, 1H), 8.39(m, 1H); MS APCI+ m/z 414 [MH]⁺

### EXAMPLES 44 AND 45

The following compounds of the general formula shown below were prepared from the product of preparation 17 using a similar method to example 43:

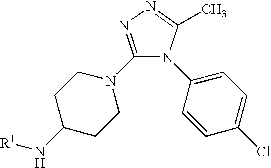

| No. | R¹ | Data | Yield |
|---|---|---|---|
| 44 | 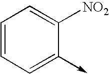 | ¹H NMR(CDCl₃, 400MHz) δ: 1.55(m, 2H), 2.06(m, 2H), 2.24(s, 3H), 2.97(m, 2H), 3.36(m, 2H), 3.59(m, 1H), 6.63(m, 1H), 6.81(d, 1H), 7.27(d, 2H), 7.40(m, 1H), 7.54(d, 2H), 8.00(d, 1H), 8.15(d, 1H); MS ES+ m/z 435 [MH]⁺ | 49% |
| 45 | 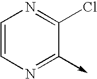 | ¹H NMR(CDCl₃, 400MHz) δ: 1.48(m, 2H), 2.02(m, 2H), 2.24(s, 3H), 2.96(m, 2H), 3.33(m, 2H), 4.00(m, 1H), 5.01(d, 1H), 7.26(d, 2H), 7.52(d, 2H), 7.55(d, 1H), 7.89(m, 1H); MS APCI+ m/z 404 [MH]⁺ | 71% |

### EXAMPLE 44

### Reaction Carried Out In Refluxing Methanol Instead of Tetrahydrofuran

### EXAMPLE 46

### N²-{1-[4-(4-Chlorophenyl)-5-methyl-4H-1,2,4-triazol-3-yl]-piperidin-4-yl}pyridine-2, 3-diamine

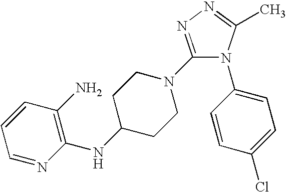

The product of example 43 (200 mg, 0.48 mmol) and Raney Nickel™ (20 mg) were added to a mixture of tetrahydrofuran (15 mL) and ethanol (7 mL) and the mixture was stirred under 30 psi of hydrogen gas for 18 hours. The reaction mixture was then filtered through glass fibre paper and the filtrate was concentrated in vacuo. The residue was azeotroped with dichloromethane to give a white solid. This solid was purified by column chromatography on silica gel, eluting with dichloromethane:methanol:0.88 ammonia, 90:10:1. The relevant fractions were concentrated in vacuo and the residue was azeotroped with dichloromethane to afford the title compound as a pale yellow solid in 96% yield, 176 mg.

¹H NMR(DMSO, 400 MHz) δ: 1.33(m, 2H), 1.84(m, 2H), 2.11(s, 3H), 2.76(m, 2H), 3.15(m, 2H), 3.90(m, 1H), 6.29(m, 1H), 6.62(d, 1H), 7.30(d, 1H), 7.52(d, 2H), 7.64(d, 2H); MS APCI+ m/z 384 [MH]⁺

### EXAMPLE 47

### N-{1-[4-(4-Chlorophenyl)-5-methyl-4H-1,2,4-triazol-3-yl]-piperidin-4-yl}benzene-1,2-diamine

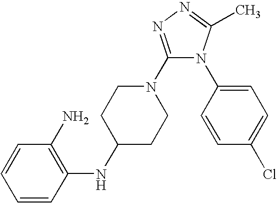

The title compound was prepared from the product of example 44, using a similar method to that of example 46, in quantitative yield.

¹H NMR(CDCl₃, 400MHz) δ: 1.40(m, 2H), 1.97(m, 2H), 2.25(s, 3H), 2.92(m, 2H), 3.28(m, 2H), 3.68(m, 1H), 6.57(m, 1H), 6.63(m, 2H), 6.72(m, 1H), 7.27(d, 2H), 7.51(d, 2H); MS ES+ m/z 405 [MH]⁺

### EXAMPLE 48

### 2-({1-[4-(4-Chlorophenyl)-5-(methoxymethyl)-4H-1,2,4-triazol-3-yl]piperidin-4-yl}oxy)pyrimidine

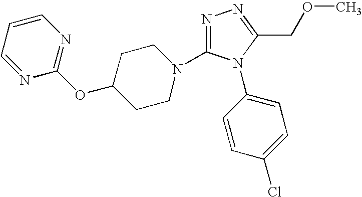

A mixture of the product of preparation 31 (200 mg, 0.55 mmol) and methoxyacethydrazide (57 mg, 0.55 mmol) in ⁿbutanol (2 mL) was heated at 140° C. for 18 hours. The mixture was then cooled to room temperature, diluted with ethyl acetate and washed with water and brine. The organic solution was dried over magnesium sulfate, concentrated in vacuo and the residue was purified by column chromatography on silica gel, eluting with dichloromethane:methanol:0.88 ammonia, 90:10:1, to afford the title compound in 45% yield, 100 mg.

¹H NMR(CD₃OD, 400MHz) δ: 1.78(m, 2H), 2.00(m, 2H), 3.04(m, 2H), 3.24(s, 3H), 3.32(m, 2H), 4.33(s, 2H), 5.09(m, 1H), 7.05(m, 1H), 7.57(d, 2H), 7.64(d, 2H), 8.52(d, 2); MS ES+ m/z 401 [MH]⁺

### EXAMPLE 49

### 2-{1-[4-(4-Chloro-phenyl)-5-methyl-4H-[1,2,4]triazol-3-yl]-piperidin-4-ylamino}-benzonitrile

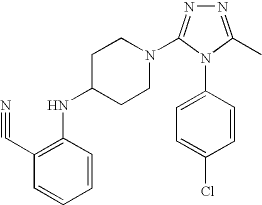

The piperidine of preparation 9 (830 mg, 2.85 mmol) was mixed with 2-fluoro-benzonitrile (3.45 g, 28.5 mmol) and potassium carbonate (565 mg, 5.7 mmol) in 1-methyl-2-pyrrolidinone (5 ml) and was heated at 120° C. for 18 hours. The reaction mixture was cooled to room temperature and was partitioned between water and ethyl acetate. The organic phase was washed three times with water, then brine, then dried over magnesium sulphate, filtered and the filtrate evaporated under reduced pressure. The residue was purified by chromatography on silica gel using a gradient of methanol, from 0 to 5%, in dichloromethane as eluant. The isolated solid was triturated with diethyl ether to give the title compound as an off-white solid (540 mg).

¹H NMR (400 MHz, CDCl₃): δ 1.44 (m, 2H), 2.03 (d, 2H), 2.16 (s, 3H), 2.92 (t, 2H), 3.38 (d, 2H), 3.47 (m, 1H), 4.38 (d, 1H), 6.66 (m, 2H), 7.31 (m, 2H), 7.38 (m, 2H), 7.67 (d, 2H); APCI: m/z 393 [MH]⁺

### EXAMPLE 50

### 2-{1-[4-(4-Chloro-phenyl)-5-methyl-4H-[1,2,4]triazol-3-yl]-piperidin-4-ylamino}-benzamide

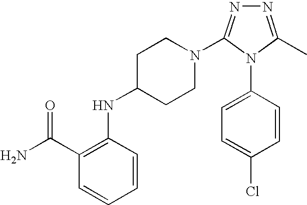

The benzonitrile of example 49 (450 mg, 1.15 mmol) was dissolved in dioxan (15 ml) and 6N aqueous sodium hydroxide (2 ml, 11.5 mmol) was added. The mixture was heated to 100° C. for 48 hours. Further 6N aqueous sodium hydroxide (2 ml, 11.5 mmol) was added and the heating continued for a further 48 hours. The reaction mixture was cooled to room temperature and partitioned between 2N aqueous sodium hydroxide and ethyl acetate. The organic phase was washed with brine, dried over magnesium sulphate, filtered and the filtrate evaporated under reduced pressure. The residue was purified by triturating with diethyl ether to give the title compound as a yellow solid (363 mg).

¹H NMR (400MHz, CD₃OD): δ 1.42 (m, 2H), 1.97 (d, 2H), 2.13 (s, 3H), 2.95 (t, 2H), 3.28 (m, 2H), 3.53 (m, 1H), 6.57 (t, 1H), 6.77 (d, 1H), 7.24 (t, 1H), 7.51 (m, 3H), 7.64 (d, 2H); APCI: m/z 411 [MH]⁺.

### EXAMPLE 51

### 3-[3-(3-Chloro-phenoxy)-azetidin-1-yl]-4-(4-chloro-phenyl)-5-methyl-4H-[1,2,4]triazole

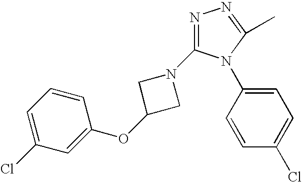

780 mg of the compound from preparation 35 (2.12 mmoles, 1 eq.), 470 mg (6.37 mmoles, 3 eq.) of acetic acid hydrazide and 250 mg (4.25 mmoles, 2 eq.) of acetic acid were dissolved in 15 ml of n-butanol, and the solution was heated at reflux for three days. The solvent was then removed under reduced pressure, and the residue was purified by flash chromatography using a gradient of DCM and DCM:MeOH (95:5 v/v), affording 319 mg (39%) of the title compound.

¹HNMR(CDCl₃, 400 MHz): 2.20 (s, 3H), 3.90(m, 2H), 4.10(m, 2H), 4.90 (m, 1H), 6.55 (m, 1H), 6.62 (m, 1H), 7.00 (m, 1H), 7.20 (m, 1H), 7.25 (m, 2H), 7.50 (m, 2H); MS APCI+ m/z 317 [MH]⁺ 375 (plus isotopic peaks).

All of the compounds of the Examples have been tested in the assays described above and found to have a Ki value of less than 500 nM.

Examples of specific test results are illustrated below

TABLE 2

| Example No. | Ki (nM) |
|---|---|
| 26 | 3.19 |
| 33 | 1.28 |
| 34 | 1.06 |
| 38 | 0.96 |
| 40 | 0.50 |
| 41 | 1.49 |

1. A compound of formula (I): 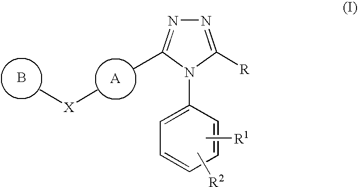
or a pharmaceutically acceptable derivative thereof, wherein
R represents C₁₋₆alkyl (optionally substituted by C₁₋₆alkyloxy or Het), or C₁₋₆alkyloxy;
R¹ and R² independently represent hydrogen, halo or C₁₋₆alkyl;
ring A represents Het¹;
X represents O or NR³;
R³ represents hydrogen or C₁₋₆alkyl;
ring B represents a phenyl group or Het², either of which may be optionally substituted with one or more groups selected from halo, CN, C₁₋₆alkyloxy, CF₃, C₁₋₆alkyl, NH₂ and NO₂;
Het and Het¹ independently represent a 5- or 6-membered saturated, partially unsaturated or aromatic heterocyclic group comprising either (a) 1 to 4 nitrogen atoms, (b) one oxygen or one sulphur atom or (c) 1 oxygen atom or 1 sulphur atoms and 1 or 2 nitrogen atoms;
Het2 represent a 5- or 6-membered aromatic heterocyclic group comprising either (a) 1 to 4 nitrogen atoms, (b) one oxygen or one sulphur atom or (c) 1 oxygen atom or 1 sulphur atoms and 1 or 2 nitrogen atoms.

2. A compound according to claim 1, wherein R represents methyl, methoxy; methoxymethyl or ethoxymethyl;

3. A compound according to claim 1, wherein R represents methylene-Het and Het represents triazolyl, morpholinyl or piperidinyl;

4. A compound according to claim 1 or claim 2, wherein R¹ represents chloro;

5. A compound according to any of claims 1 to 4, wherein R² represents hydrogen or methyl;

6. A compound according to any of claims 1 to 5, wherein ring A is attached to the triazole ring via a nitrogen atom;

7. A compound according to any of claims 1 to 6, wherein ring A represents piperidinylene;

8. A compound according to any of claims 1 to 7, wherein X represents O;

9. A compound according to any of claims 1 to 7, wherein X represents NR³ and R³ represents H or Me;

10. A compound according to any of claims 1 to 9, wherein ring B represents phenyl, pyridinyl or pyrazinyl;

11. A compound according to any of claims 1 to 10, wherein ring B is mono- or di-substituted,

12. A compound according to any of claims 1 to 11, wherein the substituent or substituents on ring B are independently selected from F, Cl, CN, methyl, methoxy, CF₃, NO₂, and CONH₂;

13. A compound according to claim 1 selected from:
1-[4-(4-Chloro-phenyl)-5-methyl-4H-[1,2,4]triazole-3-yl]-4-phenoxy-piperidine;
2-({1-[4-(4-Chlorophenyl)-5-(methoxymethyl)-4H-1,2,4-triazol-3-yl]piperidin-4-yl}oxy)pyridine;
2-{1-[4-(4-Chloro-phenyl)-5-[1,2,3]triazole-2-ylmethyl-4H-[1,2,4]triazole-piperidin-4-yloxy}-pyrimidine;
2-{1-[4-(4-Chloro-phenyl)-5-ethoxy-4H-[1,2,4]triazole-3-yl]-piperidin-4-yloxy}-pyrimidine;
N-{1-[4-(4-Chlorophenyl)-5-methyl-4H-1,2,4-triazol-3-yl]-piperidin-4-yl}-N-methylpyridin-2-amine; and
N-{1-[4-(4-Chlorophenyl)-5-methyl-4H-1,2,4-triazol-3-yl]-piperidin-4-yl}-N-methylpyrimidin-2-amine

14. The use of a compound according to any of claims 1 to 13 as a medicament.

15. A method of treatment of a mammal, including a human being, to treat a disorder for which a V1a antagonist is indicated, comprising administering a therapeutically effective amount of a compound according to any of claims 1 to 13.

16. A method of treatment of a mammal, including a human being, to treat anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittlesmerchz, preclampsia, premature ejaculation, premature (preterm) labour or Raynaud's disease, comprising administering a therapeutically effective amount of a compound according to any of claims 1 to 13 to a patient suffering from such a disorder.

17. A method of treatment according to claim 15 or claim 16, wherein the disorder is dysmenorrhoea (primary or secondary).

18. Use of a compound according to any of claims 1 to 13 in the manufacture of a medicament for the treatment of a disorder for which a V1a receptor antagonist is indicated.

19. Use of a compound according to any of claims 1 to 13 in the manufacture of a medicament for the treatment of anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittlesmerchz, preclampsia, premature ejaculation, premature (preterm) labour or Raynaud's disease.

20. Use according to claim 18 or claim 19, wherein the disorder is dysmenorrhoea (primary or secondary).

21. A pharmaceutical formulation including a compound according to any of claims 1 to 13, together with a pharmaceutically acceptable excipient, diluent or carrier.

22. A combination of (A) a compound according to any of claims 1 to 13, and (B) another pharmacologically active ingredient.

23. A combination according to claim 22, wherein (B) is an oral contraceptive, PDEV inhibitor, COX inhibitor, NO-donor or L-arginine.

24. Use of a combination according to claim 22 or claim 23, for the manufacture of a medicament for combination therapy by simultaneous, sequential or separate administration, in the treatment of dysmenorrhoea.

25. A method of treating dysmenorrhoea comprising administering to a subject in need of such treatment a combination of amounts of (A) and (B) according to claim 22 or claim 23, which are together effective.

26. A pharmaceutical product containing a combination of (A) and (B) according to claim 22 or claim 23, as a combined preparation for simultaneous, separate or sequential use in treating dysmenorrhoea (Primary or secondary).
